SEQUENCE LISTING (8 sequences)

SEQ ID NO: 1 - DNA, 3804 bp, Homo sapiens
  CDS (223)...(885)
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaggaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatgcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaatggagataatggttacaaatgtctcttcctatagtataatctccataagggcatggcccaagtctgtctttgactctgcctatccctgacgtttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccatatcgaaaatactgtactttggttgatttttaagtgggcttccattccatggatttaatcagtcccaagaagatcaaactcagcagtacttgggtgctgaagaactgttggatttaccctggcacgtgtgccacttgcccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactact

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 42 aa, Homo sapiens
IWTNSRRLLQSDYMNMTPRRPGLTRKPYQPYAPARDFAAYRP

SEQ ID NO: 4 - protein, 5 aa, Homo sapiens
IWTNS

SEQ ID NO: 5 - protein, 26 aa, Homo sapiens
IWTNSRRLLQSDYMNMTPRRPGLTRK

SEQ ID NO: 6 - protein, 42 aa, Homo sapiens
IWTNSRRLLQSDFMNMTPRRPGLTRKPYQPYAPARDFAAYRP

SEQ ID NO: 7 - protein, 42 aa, Homo sapiens
IWTNSRRLLQSDYMNMTPRRPGLTRKPYQAYAAARDFAAYRP

SEQ ID NO: 8 - protein, 42 aa, Homo sapiens
IWTNSRRLLQSDYMNMTPRRPGLTRKPYQPFAPARDFAAYRP

(19) United States
(12) Patent Application Publication
(10) Pub. No.: US 2003/0166502 A1
(43) Pub. Date: Sep. 4, 2003

(54) Differential regulation of T cell survival and proliferation

(75) Inventors: Jonathan M. Green, Creve Coeur, MO (US); Andrey S. Shaw, Saint Louis, MO (US)

Correspondence Address: LAHIVE & COCKFIELD, 28 STATE STREET, BOSTON, MA, 02109, US

(73) Assignee: Washington University, St. Louis, MO (US)

(21) Appl. No.: 10/143,238

(22) Filed: May 10, 2002

Related U.S. Application Data
(60) Provisional application No. 60/290,097, filed on May 10, 2001.

(51) Int. Cl. A61K 31/00; A61K 38/00

(52) U.S. Cl. 514/1; 514/12

(57) ABSTRACT

The present invention provides methods for selectively regulating survival and proliferation in a T cell by modulating the activity of certain amino acid motifs in the CD28 protein.

### RELATED APPLICATIONS

This application claims priority to U.S. Provisional Application 60/290,097, filed May 10, 2001, the contents of which are incorporated herein by reference.

### GOVERNMENT SUPPORT

This work was supported in part by NIH grants RO1 HL62683 and RO1 A134094, thus the government may have certain rights to the invention.

### BACKGROUND OF THE INVENTION

T lymphocytes are an essential part of the immune system. Their generation, activation, proliferation, and survival are subject to tight regulation by several extracellular factors including cytokines, MHC-antigen complexes and co-stimulatory ligands. The balanced interplay between these factors determines the fate of the T cell (Medema, J. P., and Borst, J. (1999) Hum Immunol 60, 403).

The control of peripheral T cell survival is one consequence of such interplay and is critical to the maintenance of an effective peripheral immune repertoire. Some aspects of T cell survival appear to be linked to the state of T cell activation. T cell activation is initiated by the engagement of the T cell receptor/CD3 complex (TCR/CD3) by a peptide-antigen bound to a major histocompatibility complex (MHC) molecule on the surface of an antigen-presenting cell (APC) (Schwartz, R. H. (1990) Science 248, 1349). While this is the primary signal in T cell activation, other receptor-ligand interactions between APCs and T cells are required for complete activation. For example, TCR stimulation in the absence of other molecular interactions can induce a state of anergy, such that these cells can not respond to full activation signals upon restimulation (Schwartz, R. H. (1990) Science 248, 1349; Harding, F. A., et al., (1992) Nature 356, 607). Alternatively, T cells have been shown to die by programmed cell death (PCD) when activated by TCR engagement alone (Webb, S., et al., (1990) Cell 63, 1249; Kawabe, Y., and Ochi, A. (1991) Nature 349, 245,; Kabelitz, D., and Wesselborg, S. (1992) Int. Immunol 4, 1381; Groux, H., et al., (1993) Eur. J Immunol. 23, 1623).

There are multiple receptor-ligand interactions which take place between the T cell and the APC. Many interactions are adhesive in nature and reinforce the contact between the two cells (Springer, et al., (1987) Annul. Rev. Immunol. 5, 223), while other interactions transduce additional activation signals to the T cell (Bierer, B. E., and Burakoff, S. J. (1991) Adv. Cancer Res. 56, 49). CD28, a surface glycoprotein present on 80% of peripheral T cells in humans, has been shown to be an important costimulatory receptor (June, C. H., et al., (1994) ImmunoL Today 15, 321; Linsley, P. S. and Ledbetter, J. A. (1993) Annu. Rev. Immunol. 11, 191).

Costimulation of T cells has been shown to affect multiple aspects of T cell activation (June, C. H., et al., (1994) supra) including both T cell survival and proliferation. The complicated array of second messengers induced upon CD28 engagement has made it difficult to determine whether proliferation and survival can be differentially regulated and, if so, how. Methods of selective regulation of T cell survival relative to proliferation and vice versa would be of great benefit.

### SUMMARY

The instant invention is based, at least in part, on the identification of distinct amino acid motifs within the cytoplasmic tail of the CD28 protein that selectively regulate T cell survival or T cell proliferation. Using retroviral gene transfer into primary T cells from TCR transgenic, CD28-deficient mice a specific requirement for discrete regions of the cytoplasmic tail of CD28 in the regulation of T cell proliferation and induction of the anti-apoptotic protein BCl-X_{L} has been identified. Mutation of specific proline residues abrogated the proliferative and cytokine regulatory features of CD28 costimulation. In contrast, substitution of a tyrosine important in phosphatidyl inositol 3-kinase activation left proliferation intact but prevented induction of BCl-X_{L}. Thus, the pro-survival and proliferative functions of CD28 map to distinct regions of CD28, suggesting that independent signaling cascades modulate these biologic effects.

Accordingly, one aspect of the present invention relates to a method for selectively modulating T cell survival relative to T cell proliferation. The method comprises contacting a T cell expressing a CD28 protein with an agent that selectively modulates the activity of a CD28 survival motif relative to the activity of a CD28 proliferation motif, to thereby selectively modulate survival of the T cell.

In one embodiment the agent induces or enhances phosphorylation of the CD28 protein at a tyrosine residue which corresponds to the tyrosine at position 170 of the CD28 proteins set forth in SEQ ID NO: 2.

In another embodiment said contacting step results in a detectable increase in BCl-X_{L} expression in the T cell.

In another embodiment, said modulation of T cell survival is enhancement of T cell survival.

In yet another embodiment, the agent diminishes or interferes with phosphorylation of the CD28 protein at a tyrosine residue which corresponds to the tyrosine at position 170 of the CD28 protein set forth in SEQ ID NO: 2.

In another embodiment, said contacting step results in a detectable decrease in Bcl-X_{L} expression in the T cell.

In another embodiment, said modulation of T cell survival is reduction of T cell survival.

In another embodiment, the agent is a small molecule.

In yet another embodiment, the agent acts intracellularly to selectively inhibit or interfere with phosphorylation of the CD28 molecule at a tyrosine which corresponds to the tyrosine at position 170 of the CD28 protein set forth in SEQ ID NO: 2.

In another embodiment the agent is selected from the group consisting of: an intracellular antibody, a phosphatase, and a small molecule.

Said contacting step of any of the methods described herein may occur in vivo or in vitro.

Another aspect of the present invention relates to a method for treating a subject having a condition that would benefit from selective enhancement of T cell survival relative to T cell proliferation. The method comprises providing an agent that selectively increases the activity of a CD28 survival motif relative to the activity of a CD28 proliferation motif, and administering the agent to the subject, to thereby selectively increase survival of the T cells of the subject relative to proliferation of the T cells of the subject, to thereby treat the condition.

In one embodiment, the condition is selected from the group consisting of: an immunosuppressive disorder, a condition that would benefit from survival of memory cells, and a neoplasia.

In another embodient, the agent is selected from the group consisting of: an antibody and a small molecule that acts intracellularly to induce or enhance the phosphorylation of the CD28 protein at a tyrosine which corresponds to the tyrosine at position 170 of CD28 protein set forth in SEQ ID NO: 2.

In another embodiment, the method further comprises administering a second agent to the subject, wherein the second agent has immunostimulatory activity.

In another embodiment, the agent acts intracellularly on the T cells of the subject to induce or enhance the interaction of the CD28 survival motif with a CD28 survival motif binding partner.

In another embodiment the CD28 survival motif binding partner is PI-3 kinase, or is alternatively selected from the group consisting of PI-3 kinase, Grb-2, and Gads.

In anther embodiment, the agent of acts intracellularly on the T cells of the subject to induce or enhance the phosphorylation of CD28 at a tyrosine which corresponds to the tyrosine residue at position 170 of the CD28 protein set forth in SEQ ID NO: 2.

In another embodiment, the step of administering results in a detectable increase in the expression of BCl-X_{L} protein.

In another embodiment, the agent is selected from the group consisting of an antibody and a small molecule.

Another aspect of the present invention relates to a method for treating a subject having a condition that would benefit from selective reduction of T cell survival relative to T cell proliferation. The method comprises administering an agent that selectively decreases the activity of a CD28 survival motif relative to the activity of a CD28 proliferation motif to the subject to deliver the agent intracellularly to T cells of the subject, to thereby selectively decrease survival of the T cells of the subject relative to proliferation of the T cells of the subject, to thereby treat the condition.

In one embodiment, the agent acts intracellularly on the T cells of the subject to inhibit the interaction of the CD28 survival motif with a CD28 survival motif binding partner.

In another embodiment, the agent acts intracellularly on the T cells of the subject to diminish or interfere with the phosphorylation of a tyrosine residue at position 170 of the CD28 protein.

In yet another embodiment, the step of administering results in a detectable decrease in the expression of BCl-X_{L} protein in the T cells of the subject.

In another embodiment, the method further comprises administering a second agent to the subject, wherein the second agent is an immunodepressant.

In another embodiment, the agent is selected from the group consisting of: a phosphatase, an antibody, and a small molecule.

In one embodiment, the condition is selected from the group consisting of a T cell neoplasia, a transplant-associated disorder, an allergic disease, graft-versus-host disease, and an autoimmune disease.

Another aspect of the present invention relates to a cell-based method for identifying a compound which selectively modulates T cell survival relative to T cell proliferation. The method comprises contacting a cell which expresses a CD28 protein that comprises a CD28 survival motif, in the context of a cell-based assay, with a test compound and thenassaying for the ability of the test compound to modulate the phosphorylation of CD28 at a tyrosine residue which corresponds to the tyrosine at position 170 of the CD28 protein set forth in SEQ ID NO: 2. A determination that the test compound modulates the phosphorylation of the CD28 protein, indicates that the test compound is a compound which selectively modulates T cell survival.

Another aspect of the present invention relates to a cell-based method for identifying a compound which selectively modulates T cell survival relative to T cell proliferation. The method comprises contacting a cell which expresses a CD28 protein that comprises a CD28 survival motif, in the context of a cell based assay, with a test compound, and assaying for the ability of the test compound to modulate the activity or expression of BCl-X_{L} in the cell. A determination that the test compound modulates the activity or expression of BCl-X_{L} in the cell indicates that the test compound is a compound which selectively modulates T cell survival.

Another aspect of the invention relates to a cell-free method for identifying a compound which selectively modulates T cell survival relative to T cell proliferation. The method comprises contacting a polypeptide comprising a CD28 survival motif in the context of a cell-free assay system, with a test compound, and assaying for the ability of the test compound to modulate phosphorylation of the CD28 survival motif at a tyrosine that corresponds to the tyrosine at position 170 of CD28 set forth in SEQ ID NO: 2, by measuring a readout. The readout may be (i) test compound binding to the region of the polypeptide comprising the tyrosine residue or (ii) test compound modulation of phosphorylation of CD28 survival motif at the tyrosine residue. Detection of either readout (i) or readout (ii) indicates that the test compound is a compound that selectively modulates T cell survival.

Another aspect of the invention relates to a method for selectively modulating T cell proliferation relative to T cell survival. The method comprises contacting a T cell expressing a CD28 protein with an agent that selectively modulates the activity of a CD28 proliferation motif relative to the activity of a CD28 survival motif, to thereby selectively modulate proliferation of the T cell relative to survival of the T cell.

In one embodiment, the agent selectively modulates the activity of a CD28 proliferation motif by modulating the interaction of prolines corresponding to the proline at position 187 and the proline at position 190 of the CD28 protein set forth in SEQ ID NO: 2, with a molecule with which the CD28 proliferation motif interacts.

In another embodiment, the molecule is Lck protein.

In another embodiment, the contacting step results in an induction or enhancement of CD28 protein interaction with the SH3 domain of Lck protein.

In another embodiment, the contacting step results in a detectable increase in the expression of IL-2 protein.

In another embodiment, the modulation of T cell proliferation is enhancement of T cell proliferation.

In another embodiment, the contacting step results in a detectable diminishment or interference with the interaction of the SH3 domain of Lck and the CD28 protein

In another embodiment, the contacting step results in a detectable decrease in the expression of IL-2 protein by the T cell.

In another embodiment, modulation of T cell proliferation is reduction in T cell proliferation.

In yet another embodiment, modulation of T cell proliferation is enhancement of T cell proliferation, and the agent is a small molecule which induces or increases the interaction.

In another embodiment, modulation is inhibition of T cell proliferation, and the agent inhibits said interaction wherein said agent is an antibody or a small molecule.

The step of contacting in any of the above described methods may occur in vivo or in vitro.

Another aspect of the invention relates to a method for treating a subject having a condition that would benefit from selective increase of T cell proliferation relative to T cell survival. The method comprises administering to the subject an agent that acts intracellularly to induce or enhance interaction of the prolines of CD28 protein which correspond to the proline at position 187 and the proline at position 190 of the CD28 protein set forth in SEQ ID NO: 2 with a molecule with which the CD28 proliferation motif interacts. Administering results in the delivery of the agent intracellularly to T cells of the subject, to selectively increase proliferation of the T cells.

In one embodiment, the administering step results in a detectable increase in IL-2 protein production of the T cells of the subject.

In another embodiment, the agent is a small molecule, and the molecule is Lck. In another embodiment, the method further comprises administering a second agent to the subject wherein the second agent is an immunostimulant.

In one embodiment, the condition is a neoplasia, or an immunosuppressive disease.

Another aspect of the present invention relates to a method for treating a subject having a condition that would benefit from selective reduction of T cell proliferation relative to T cell survival. The method comprises administering to the subject an agent that acts intracellularly to diminish or interfere with interaction of the prolines of the CD28 protein which correspond to the proline at position 187 and the proline at position 190 of the CD28 protein set forth in SEQ ID NO: 2, with a molecule which interacts 30 with a CD28 proliferation motif. Administration to the subject delivers the agent intracellularly to T cells of the subject, to thereby selectively reduce proliferation of the T cells.

In one embodiment, administering results in a detectable decrease in IL-2 production of the T cells of the subject.

In another embodiment, the molecule is Lck protein, and the agent is an intracellular antibody, a dominant negative mutant, an antisense oligonucleotide, or a small molecule.

In one embodiment, the method further comprises administering a second agent to the subject, wherein the second agent is an immunosuppressant.

In one embodiment, the condition a T cell neoplasia, an allergic disease, a transplant-associated disorder, graft-versus-host disease, or a lymphoproliferative disorder.

Another aspect of the invention relates to a cell-based method for identifying a compound which selectively modulates T cell proliferation relative to T cell survival. The method comprises contacting a cell, which expresses a CD28 protein that comprises a CD28 proliferation motif, in the context of a cell-based assay with a test compound, and assaying for the ability of the test compound to modulate the interaction of the prolines of the CD28 proliferation motif, which correspond to prolines 187 and 190 of the CD28 protein set forth in SEQ ID NO: 2, with a molecule with which the CD28 proliferation motif interacts. A determination that the test compound modulates the interaction of the prolines of the CD28 proliferation motif with a molecule with which the CD28 proliferation motif interacts, indicates that the test compound is a compound which selectively modulates T cell proliferation relative to T cell survival.

In one embodiment, the assaying step comprises measuring the ability of the test compound to modulate the expression of IL-2 by the T cells.

Another aspect of the present invention relates to a cell-free method for identifying a compound which selectively modulates T cell proliferation relative to T cell survival. The method comprises contacting a polypeptide comprising a CD28 proliferation motif, in the context of a cell-free assay system with a test compound, and assaying for the ability of the test compound to modulate the interaction of the prolines of the CD28 proliferation motif which correspond to prolines 187 and 190 of the CD28 protein set forth in SEQ ID NO: 2, with a molecule with which the CD28 proliferation motif interacts. A determination that the test compound modulates the interaction of the prolines of the CD28 proliferation motif with a molecule with which the CD28 proliferation motif interacts, indicates that the test compound is a compound which selectively modulates T cell proliferation relative to T cell survival.

### BRIEF DESCRIPTION OF THE DRAWINGS

FIG. 1 is a graphical representation of the effect of expression of CD28 on retrovirally infected T cells: A) The amino acid sequence of wild type and mutant CD28 proteins expressed are shown: CD28 wild type (SEQ ID NO: 3); CD28dcyto (SEQ ID NO: 4); CD28d16 (SEQ ID NO: 5); CD28Y170F (SEQ ID NO: 6); CD28P187,190A (SEQ ID NO: 7); CD28Y188F (SEQ ID NO: 8). Specific substitutions are shown in bold type. B) In the experiment, splenocytes from CD28+/+or CD28 −/−DO 11.10 mice were infected with retrovirus encoding for control (GFPRV) or CD28 protein (CD28RV). Expression of CD28 was determined by staining with PE conjugated anti-CD28 mAb. Shown is a histogram plot of the PE fluorescence after, gating on GFP positive cells. All CD28 mutants exhibited expression similar to endogenous CD28 expression on wild type cells (CD28+/++GFPRV) indicated as overlapping histograms labeled CD28 −/−+CD28RV.

FIG. 2 contains graphical representations of the proliferation of T cells which express the various CD28 mutants, in response to antigen presentation. Splenocytes from CD28-deficient DO 11.10 mice were infected with retrovirus encoding either control (GFPRV), wild type CD28 (FLCD28) or mutant CD28 constructs, CD28d16, CD28P187, 190A (P187, 190A), and CD28Y188F (Y188F). Proliferation was determined following stimulation with OVA₍₃₂₃₋₃₃₉₎ peptide and irradiated T-depleted splenocytes from Balb/c mice. Either CTLA4Ig or a control Ig was added at the initiation of the culture. Addition of the control Ig had no effect on proliferation. Replicate wells were activated with PMA (5 ng/ml) and lonomycin (0.1 μg/ml). Cell proliferation was determined by tritiated thymidine incorporation for the final 8 hours of a 72 hour culture. Data from one representative experiment is shown. The data indicates that CD28-dependent proliferation requires that the CD28 protein contain prolines 187 and 190.

FIG. 3 contains three bar graphs. Splenocytes from CD28-deficient DO11.10 mice were infected with retrovirus encoding either control (GFPRV) or wild type CD28 (FLCD28) or mutant CD28 constructs and stimulated with OVA₍₃₂₃₋₃₃₉₎ peptide alone, with CTLA4Ig (10 μg/ml) or with anti-CD28 mAb (1.0 μg/ml). Culture supernatant was harvested at 48 hours and assessed for IL-2 by CTLL-2 bioassay. Shown is the tritiated thymidine incorporation of the CTLL-2 cells. The mean +/−the std deviation of quadruplicate wells is plotted. Data from one representative experiment is shown. A) CTLL-2 assay following stimulation with 0.1 μM OVA. B) CTLL-2 assay following stimulation with 0.03 μM OVA. The data indicates that CD28 regulation of IL-2 requires prolines 187 and 190 of the CD28 protein.

FIG. 4 is a photo of Western blots of cell extracts from A) Splenocytes from CD28-deficient DO11.10 mice infected with retrovirus encoding either control (CD4RV), wild type CD28 (FLCD28) or the indicated mutant CD28 constructs and stimulated with plate bound anti-CD3 (10 μg/ml) and soluble anti-CD28 (1.0 μg/ml) for 48 hours; B) CD28-deficient splenocytes retrovirally infected with either wild type CD28 (FLCD28) or the CD28P187,190A mutant and stimulated as in A) with or without CTLA4Ig (10 μg/ml). The cells were lysed in 0.2 % NP40 lysis buffer and the protein separated on a 12.5 % SDS-PAGE gel and transferred to a PVDF membrane. The membranes were probed with anti-Bcl-X_{L} anti-sera followed by HRP conjugated goat anti-rabbit Ig and developed by ECL. Subsequently, the blots were stripped and reprobed with anti-actin mAb to verify equal protein loading. The data indicate induction of Bcl-X_{L} by CD28 requires a tyrosine at position 170 of CD28.

FIG. 5 contains histogram plots of (top panel) CD69 expression of the CD4 positive population by unstimulated cells, cells stimulated with OVA peptide antigen or OVA peptide antigen plus the PI 3-kinase inhibitor LY294002; (bottom panel) Bcl-X_{L} expression of the CD4 positive population by unstimulated cells, cells stimulated with OVA peptide antigen or OVA peptide antigen plus the PI 3-kinase inhibitor LY294002. The data shows that PI 3-kinase activity is required for induction of Bcl-XL.

### DETAILED DESCRIPTION

T cell survival and proliferation are both regulated by CD28 engagement. The instant invention is based, at least in part, on the identification of distinct amino acid motifs within the cytoplasmic tail of the CD28 protein that selectively regulate T cell survival or T cell proliferation. While CD28 activation was known to affect both cell proliferation and survival, the results of experiments detailed in the Examples section below indicate that these two effects, proliferation and survival, are distinct and separable. As such, the present invention relates to a method of independently regulating cell proliferation and cell survival which results from CD28 activation.

In Examples 1-3, primary T cells from TCR transgenic, CD28-deficient mice were reconstituted with wild type or mutant CD28 in order to examine the structural features of CD28 in a physiologic context, more specifically the role of CD28 in activation of T cells with peptide antigen presented by normal APC. Deletion of the C-terminal 16 amino acids of CD28 led to a complete loss of the ability of CD28 to augment both proliferation and IL-2 secretion in response to activation. Mutation of the proline residues in the PYAP motif found in this region was sufficient to abrogate CD28-mediated proliferation and IL-2 secretion. In contrast, mutation of the tyrosine within this motif had no effect on CD28-mediated proliferation or IL-2 secretion. These results indicated that CD28-mediated proliferation and cytokine secretion is mediated by recruitment of other proteins to CD28 via interaction of the PYAP motif of CD28 with SH3 domains of other proteins.

Also present in the C-terminus of CD28 is a membrane proximal YMNM motif, which has been demonstrated to bind and activate PI-3 kinase. Previously, the role of this region in CD28 function in the art was inconclusively determined, with some research groups demonstrating a requirement for the motif in CD28-mediated proliferation and cytokine secretion, and other groups reporting that the motif was not necessary. In Example 1, reconstitution of CD28-deficient T cells with a mutant CD28 which has the tyrosine of this motif replaced by a phenylalanine had a very moderate defect on proliferation induction. IL-2 secretion induced by this mutant was identical to that of wild type CD28.

As discussed above, activation of CD28 influences cell survival by altering susceptibility to apoptosis, through induction of the anti-apoptotic protein Bcl-X_{L}. Mutation of the tyrosine at position 170 prevents CD28 dependent upregulation of Bcl-X_{L}, while having no effect on T cell proliferation. Thus, modulation of phosphorylation of the tyrosine at position 170 of the CD28 protein can selectively modulate cell survival relative to proliferation; modulation of the interaction between CD28 and SH3 domain containing proteins which are recruited to CD28 via interaction with proline 187 and proline 190 of CD28 (e.g., Lck) selectively modulates T cell proliferation relative to survival.

I. Definitions

As used herein, the various forms of the term “modulate” are intended to include stimulation (e.g., increasing or upregulating a particular response or activity) and inhibition (e.g., decreasing or downregulating a particular response or activity). Modulation is reproducibly detectable by one or more means known in the art and is to a degree which is statistically significant. Modulation may be by direct or indirect means.

The term “T cell” is art-recognized and includes thymocytes, immature T lymphocytes, mature T lymphocytes, resting T lymphocytes, and/or activated T lymphocytes. Preferably, the term “T cell” includes primary T cells and excludes transformed T cells (e.g., T cells that do not require repeated receptor-mediated stimulation in order to proliferate, such as Jurkat cells). A T cell can be a T helper (Th) cell, for example a T helper 1 (Th1) or a T helper 2 (Th2) cell or the cell can be a cytotoxic T cell. The T cell can be a CD4+T cell, CD8+T cell, CD4+CD8+T cell, CD4−CD8−T cell, or any other subset of T cells.

The term “CD28 antigen” as used herein refers to the cluster of differentiation antigen exposed on T cells which serves as a receptor for binding molecules of the B7 family (e.g., B7.1 and B7.2), a homodimeric glycoprotein of the immunoglobulin superfamily (Aruffo and Seed, Proc. Natl. Acad. Sci. 84:8573-8577 (1987)) which is found on most mature human T cells (Damle et al., J. Immunol. 131:2296-2300 (1983). “CD28 protein,” also refered to as simply “CD28,” comprises approximately 199 amino acid residues. The “cytoplasmic tail” of CD28 comprises amino acids 158 to 199. The nucleotide sequence encoding human CD28 and the amino acid sequences of human CD28 are shown in SEQ ID NO: 1 and SEQ ID NO: 2, respectively.

As used herein, the term “CD28 survival motif” includes the tyrosine amino acid residue at position 170 of the wild-type CD28 molecule. The CD28 survival motif may further include adjacent amino acids. In one embodiment, the motif is YMNM. In another embodiment, the motif includes 3-5 amino acids flanking the tyrosine at position 170. In another embodiment, the motif includes 6-10 amino acids flanking the tyrosine at position 170. As used herein, the term “CD28 survival motif activity” includes, e.g., phosphorylation of the tyrosine at position 170 the ability of the motif to regulate BCl-X_{L} expression, the ability of the motif to regulate cell survival.

As used herein, the term “CD28 proliferation motif” includes the proline at position 187 and the proline at position 190 of the wild-type CD28 molecule. The motif may also include adjacent amino acids. In one embodiment, the motif includes 3-5 amino acids flanking the respective prolines at position 187 and 190. In another embodiment, the motif includes 6-10 amino acids flanking the respective prolines at position 187 and 190. As used herein, the term “CD28 proliferation motif activity” includes, e.g., binding of the proline at position 187 and 190 to bind to an SH3 domain contained within another protein (e.g., the SH3 domain within the protein Lck) and also the ability of the motif to regulate cell proliferation. As used herein, the term “CD28 activity” includes CD28 proliferation and/or CD28 survival motif activity.

Polypeptides comprising these CD28 motifs include the entire wild-type CD28 protein or a portion thereof which comprises the motif. In addition, polypeptides comprising a CD28 survival or proliferation motif can be variants of a CD28 protein or a portion thereof, or peptidomimetics including such a motif. Exemplary portions of CD28 molecules include the cytoplasmic tail of CD28. In one embodiment, a CD28 motif described herein can comprise non-CD28 sequences at the COOH and/or NH2 terminus.

As used herein, the term “Src Homology Domain3”(SH3) refers to src homology domains of a protein that have homology with src and are involved in the interaction with proline rich sections of other proteins. “Lck” is a nonreceptor tyrosine kinase, related to src. Lck is expressed exclusively in lymphoid cells, most predominantly in thymocytes (Luo, K., and Sefton, B. M. (1990) Mol. Cell. BioL 10, 5305-5313).

As used herein, the term “wild-type” here is referred to the naturally occurring, native, or non-mutated version of a gene, e.g., the CD28 gene. It is also used to refer to the message or protein encoded by the native or non-mutated gene.

As used herein, a “naturally-occurring” nucleic acid molecule refers to an RNA or DNA molecule having a nucleotide sequence that occurs in nature (e.g., encodes a natural protein).

As used herein, the language “selectively regulating T cell survival” refers to modulating (e.g., inhibiting, delaying, or enhancing) cell survival (i.e., cell viability) without significantly regulating T cell proliferation. The terms cell survival refers to the ability of a cell to survive death occurring via a number of different mechanisms, e.g., programmed cell death (PCD), “apoptosis,” and/ or “necrosis.”

As used herein the term “apoptosis” is a form of programmed cell death which can be characterized using techniques which are known in the art. Apoptotic cell death can be characterized, e.g., by cell shrinkage, membrane blebbing and chromatin condensation culminating in cell fragmentation. Cells undergoing apoptosis also display a characteristic pattern of internucleosomal DNA cleavage. As used herein, the term “modulating apoptosis” refers to modulating apoptotic programmed cell death in a cell, such as a T cell. As used herein, the term “modulates apoptosis” includes either up regulation or down regulation of T cell survival, hence, apoptosis in a cell.

As used herein, the term “selectively regulating T cell proliferation” includes modulating (e.g., inhibiting, delaying, or enhancing) cell proliferation (i.e., cell expansion or increase in cell numbers) without significantly regulating T cell survival.

The term “immunogen” is used herein to describe a composition containing a peptide or protein as an active ingredient used for the preparation of antibodies against an antigen (e.g., CD28).

As used herein, the term “antibody” is intended to include immunoglobulin molecules and immunologically active portions of immunoglobulin molecules, i.e., molecules that contain an antigen binding site which binds (immunoreacts with) an antigen, such as Fab and F(ab′)hd 2 fragments, single chain antibodies, intracellular antibodies, scFv, Fd, or other fragments. Preferably, antibodies of the invention bind specifically or substantially specifically to CD28.

The terms “monoclonal antibodies” and “monoclonal antibody composition”, as used herein, refer to a population of antibody molecules that contain only one species of an antigen binding site capable of immunoreacting with a particular epitope of an antigen. A monoclonal antibody composition thus typically displays a single binding affinity for a particular antigen with which it immunoreacts.

The term “polyclonal antibodies” and “polyclonal antibody composition” refer to a population of antibody molecules that contain multiple species of antigen binding sites capable of interacting with a particular antigen.

The term “intracellular antibody” refers to an antibody that functions in an intracellular region of a cell, e.g., the cytoplasm or nucleus. As used to refer to the present invention, an intracellular antibody serves to modulate the expression or activity of a CD28 motif. An intracellular antibody is an antibody that has been engineered such that: 1) the antigen binding domain which specifically binds (immunoreacts with) an antigen, derived from an immunoglobulin molecule or immunologically active portion of an immunoglobulin molecule, usually a monoclonal antibody (described above), expressed in recombinant form, such as a single-chain Fv fragment or Fab fragment, or F(ab′)2, with scFv and Fab′ fragments being the preferred fragments (e.g., raised against CD28 protein having a Tyrosine at position 170 and/or having proline residues at position 187 and position 190). The fragment is expressed with a bacterial leader sequence at the N-terminus responsible for export of the protein to the periplasmic space. Here, the VH and VL domains fold into active protein including the formation of the disulfide bonds present in the two domains, and 2) by using suitable expression systems, the fragment is expressed in a variety of different cells, including mammalian cells, preferably in Human T cells.

As used herein, the term “binding partner” or “motif binding partner” as it applies to a CD28 proliferation motif or a CD28 survival motif, refers to a molecule which binds either the CD28 proliferation motif or CD28 survival motif in response to activation of the CD28 molecule, to produce a downstream effect on proliferation or survival, respectively. A CD28 survival motif binding partner requires the presence of a tyrosine residue at position 170 of the CD28 protein. A CD28 proliferation motif binding partner requires the presence of a proline at position 187 and 190 of the CD28 protein.

As used herein, the term “a modulator of a CD28 survival motif” refers to an agent, for example a compound or compounds, which modulates the activity of the CD28 survival motif (e.g., via modulation of phosphorylation of a tyrosine residue at position 170 of CD28) without significantly modulating the activity of a CD28 proliferation motif (e.g., without interfering with the interaction of a CD28 proliferation motif with its binding partner(s)) or without causing a detectable change in cell proliferation).

As used herein, the term “a modulator of a CD28 proliferation motif” refers to an agent, for example, a compound or compounds, which modulates the activity of a CD28 proliferation motif (e.g., via modulating the interaction of proline at position 187 and proline at position 190 of the CD28 protein with CD28 proliferation motif binding partners, such as molecules that interact with a CD28 proliferation motif (e.g., the SH3 domain of the Lck protein), without significantly modulating a CD28 survival motif (e.g., without modulating phosphorylation of a tyrosine residue at position 170 of CD28, or causing a detectable change in cell survival.

As used herein, the term “contacting” (e.g., contacting a cell with an compound) is intended to include incubating the compound and the cell together in vitro (e.g., adding the compound to cells in culture) or administering the compound to a subject such that the compound and cells of the subject are contacted in vivo.

As used herein, the term “test compound” is intended to refer to a compound that has not previously been conclusively identified as, or recognized to be, a modulator of a CD28 survival or proliferation motif activity. This includes compounds suspected of functioning as a modulator of CD28 survival or proliferation motif activity. Preferred test compounds of the invention act intracellularly to modulate a CD28 survival or proliferation motif. A compound can be naturally found in vivo (e.g., may occur as a result of a natural physiological process). Preferably, the compound is one to which the cell it is not naturally exposed in vivo in an amount sufficient to modulate a desired biological response, e.g., proliferation or survival. In one embodiment, compounds to which a cell might be naturally exposed in vivo are excluded as test compounds.

The term “library of test compounds” is intended to refer to a panel comprising a multiplicity of test compounds.

As used herein, the term “indicator composition” refers to a composition that includes a CD28 motif, and is used to detect the activity of the CD28 motif. For example, a cell that naturally expresses CD28 protein, a cell that has been engineered to express a CD28 protein or CD28 motif by introducing an expression vector encoding the CD28 protein or CD28 motif containing portion thereof into the cell, or a cell free composition that contains a CD28 motif (e.g., naturally-occurring CD28 or recombinantly-engineered CD28 or CD28 motif containing portions thereof).

As used herein, the term “engineered” (as in an engineered cell) refers to a cell into which an expression vector (e.g., a vector encoding the CD28 protein) has been introduced.

As used herein, the term “cell free composition” refers to an isolated composition which does not contain intact cells. Examples of cell free compositions include cell extracts and compositions containing isolated proteins.

As used herein, the term “target molecule for CD28” refers a molecule with which CD28 can interact, including proteins such as, Lck.

As used herein, the term “reporter gene” refers to any gene that expresses a detectable gene product, which may be RNA or protein. Preferred reporter genes are those that are readily detectable (e.g., via a property or activity of the encoded protein). The reporter gene may also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368), and green fluorescent protein (U.S. Pat. No. 5,491,084; WO 96/23898).

As used herein, an “effective amount” of a compound is the minimum amount of a compound that is necessary to minimally achieve, and more preferably, optimally achieve, the desired effect (e.g. selective regulation T cell survival or proliferation).

As used herein, the term “interact” refers to detectable interactions between proteins or protein motifs, or between proteins or protein motifs and test compounds. Protein-protein interactions can be detected using, for example, coimmunoprecipitation assays or readout assays. The term interact is also meant to include binding interactions between molecules.

As used herein, the term “treat”, or “treatment”, as it relates to treatment of a disorder, refers to application of the methods of the invention to the treatment of a disorder which results in cure of the disorder, a decrease in the type or number of symptoms associated with the disorder, either in the long term or short term (i.e., amelioration of the condition), or simply a transient beneficial effect to the subject.

As used herein, the terms “gene” and “recombinant gene” refer to nucleic acid molecules which include an open reading frame encoding a CD28 protein, preferably a mammalian CD28 protein, and can further include non-coding regulatory sequences, and introns.

As used herein, “heterologous DNA” or “heterologous nucleic acid” refers to DNA that does not occur naturally as part of the genome in which it is present or which is present in a location or locations in the genome that differs from that in which it occurs in nature, or which is operatively linked to DNA to which it is not normally linked in nature (i.e., a gene that has been operatively linked to a heterologous promoter). Heterologous DNA is not necessarily integrated into the genome, and further includes DNA which is not endogenous to the cell into which it is introduced, but has been obtained from another cell. Heterologous DNA can be from the same species or from a different species.

The terms “heterologous protein,”“recombinant protein,” and “exogenous protein,” are used interchangeably throughout the specification to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

As used herein, the term “isolated” as used to describe a nucleic acid molecule, distinguishes the nucleic acid molecule as one which is separated from other nucleic acid molecules which are present in the natural source of the nucleic acid molecule. For example, with regards to genomic DNA, the term “isolated” specifies nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived. For example, in various embodiments, the isolated CD28 nucleic acid molecule can contain less than about 5 kb, 4kb, 3kb, 2kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. An “isolated” CD28 nucleic acid molecule may, however, be linked to other nucleotide sequences that do not normally flank the CD28 sequences in genomic DNA (e.g., the CD28 nucleotide sequences may be linked to vector sequences). It is not necessary for the nucleic acid molecule to be free of other cellular material to be considered “isolated” (eg., CD28 DNA molecule separated from other mammalian DNA and inserted into a bacterial cell would still be considered to be “isolated”).

As used herein, an “isolated protein” or “isolated polypeptide” refers to a protein or polypeptide that is substantially free of other proteins, polypeptides, cellular material and culture medium when isolated from cells or produced by recombinant DNA techniques, or chemical precursors or other chemicals when chemically synthesized. An “isolated” or “purified” CD28 protein or biologically active portion thereof is substantially free of cellular material or other contaminating proteins from the cell or tissue source from which the CD28 protein is derived, or substantially free from chemical precursors or other chemicals when chemically synthesized. The language “substantially free of cellular material” includes preparations of CD28 protein in which the protein is separated from cellular components of the cells from which it is isolated or recombinantly produced. In one embodiment, the language “substantially free of cellular material” includes preparations of CD28 protein having less than about 30% (by dry weight) of non- CD28 protein (also referred to herein as a “contaminating protein”), more preferably less than about 20% of non- CD28 protein, still more preferably less than about 10% of non- CD28 protein, and most preferably less than about 5% non- CD28 protein. When the CD28 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CD28 protein in which the protein is separated from chemical precursors or other chemicals which are involved in the synthesis of the protein. In one embodiment, the language “substantially free of chemical precursors or other chemicals” includes preparations of CD28 protein having less than about 30% (by dry weight) of chemical precursors or non- CD28 chemicals, more preferably less than about 20% chemical precursors or non- CD28 chemicals, still more preferably less than about 10% chemical precursors or non- CD28 chemicals, and most preferably less than about 5% chemical precursors or non- CD28 chemicals.

As used herein, the term “antisense” as it refers to a nucleic acid indicates that the nucleic acid comprises a nucleotide sequence which is complementary to a “sense” nucleic acid. In one embodiment, the “sense” nucleic acid encodes a protein, and thus the antisense is complementary to the coding strand of a double-stranded CDNA molecule, complementary to an mRNA sequence or complementary to the coding strand of a gene. The “sense” nucleic acid can also be non-coding nucleic acid, (e.g. regulatory sequences). Accordingly, an antisense nucleic acid can hydrogen bond to a sense nucleic acid.

II. Cells Expressing CD28 Polypeptides or Portions Thereof

A. CD28 Nucleic Acid and Polypeptide Molecules

The present invention pertains to CD28 nucleic acid molecules and polypeptides. Portions of such CD28 molecules can also be used in practicing the invention.

One aspect of the invention pertains to isolated nucleic acid molecules that encode CD28 proteins or biologically active portions thereof, as well as nucleic acid fragments sufficient for use as hybridization probes to identify CD28 -encoding nucleic acids (e.g., CD28 mRNA) and fragments for use as PCR primers for the amplification or mutation of CD28 nucleic acid molecules or portions thereof. It will be understood that in discussing the uses of CD28 nucleic acid molecules, e.g., as shown in SEQ. ID NO:1 or a nucleotide sequence encoding another CD28 polypeptide, that fragments of such nucleic acid molecules as well as full length CD28 nucleic acid molecules can be used.

A nucleic acid molecule of the present invention, e.g., a nucleic acid molerule having the nucleotide sequence of SEQ ID NO:1 or a nucleotide sequence encoding another CD28 polypeptide, or a portion thereof, can be isolated using standard molecular biology techniques and the sequence information provided herein. For example, using all or portion of the nucleic acid sequence of SEQ ID NO:1 or a nucleotide sequence encoding another CD28 polypeptide as a hybridization probe, CD28 nucleic acid molecules can be isolated using standard hybridization and cloning techniques (e.g., as described in Sambrook, J., Fritsh, E. F., and Maniatis, T. Molecular Cloning. A Laboratory Manual 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 or a nucleotide sequence encoding another CD28 polypeptide can be isolated by the polymerase chain reaction (PCR) using synthetic oligonucleotide primers designed based upon the sequence of SEQ ID NO:1 or a nucleotide sequence encoding another CD28 polypeptide respectively.

Nucleic acid sequences encoding other CD28 polypeptides can be identified based on nucleic acid and/or amino acid identity with CD28, possession of one or more CD28 domains or motifs, and/or possession of a CD28 activity, or CD28 motif activity, as defined herein.

A nucleic acid of the invention can be amplified using cDNA, mRNA or, genomic DNA as a template, and appropriate oligonucleotide primers, according to standard PCR amplification techniques. The nucleic acid so amplified can be cloned into an appropriate vector and characterized by DNA sequence analysis. Furthermore, oligonucleotides corresponding to CD28 nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:1 a nucleic acid molecule encoding another CD28 polypeptide. In a preferred embodiment, the isolated nucleic acid molecule encodes a polypeptide comprising a CD28 survival motif, as describes herein, either in isolation, or attached to heterologous polypeptide sequences. In another preferred embodiment the isolated nucleic acid molecule encodes a polypeptide comprising a CD28 proliferation motif, as described herein. The nucleic acid molecule of the present invention may further encode both motifs, in relative isolation, or attached to heterologous polypeptide sequences.

In another embodiment, an isolated nucleic acid molecule of the invention comprises a nucleic acid molecule which is a complement of the nucleotide sequence shown in SEQ ID NO:1 or a nucleotide sequence encoding another CD28 polypeptide or a portion of any of these nucleotide sequences, especially a portion encoding a proliferation or survival motif. A nucleic acid molecule which is complementary to the nucleotide sequence shown in SEQ ID NO:1 or a nucleotide sequence encoding another CD28 polypeptide is one which is sufficiently complementary to the nucleotide sequence shown in SEQ ID NO:1 or a nucleotide sequence encoding another CD28 polypeptide, or portion of either, respectively, such that it can hybridize to the nucleotide sequence shown in SEQ ID NO:1 or a nucleotide sequence encoding another CD28 polypeptide respectively, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 98% or more homologous to the nucleotide sequence (e.g., to the entire length of the nucleotide sequence) shown in SEQ ID NO:1, or a portion thereof, or a nucleotide sequence encoding another CD28 polypeptide or a portion thereof. Relevant portions include an intracellular domain, an extracellular domain, a transmembrane domain, a zinc finger domain, a glutamic acid/aspartic acid-rich domain or a serine/threonin-proline-X-arginine/lysine domain, a CD28 survival motif containing domain, and a CD28 proliferation containing domain.

Moreover, the nucleic acid molecule of the invention can comprise only a portion of the nucleic acid sequence of SEQ ID NO:1 a nucleic acid molecule encoding another CD28 polypeptide for example a fragment which can be used as a probe or primer or a fragment encoding a biologically active portion of a CD28 protein. The nucleotide sequence determined from the cloning of the CD28 genes allows for the generation of probes and primers designed for use in identifying and/or cloning yet other CD28 family members, as well as CD28 family homologues from other species. The probe/primer typically comprises a substantially purified oligonucleotide. In one embodiment, the oligonucleotide comprises a region of nucleotide sequence that hybridizes under stringent conditions to at least about 12 or 15, preferably about 20 or 25, more preferably about 30, 35, 40, 45, 50, 55, 60, 65, 75, or 100 consecutive nucleotides of a sense sequence of SEQ ID NO:1 or a nucleotide sequence encoding another CD28 polypeptide or of a naturally occurring allelic variant or mutant of SEQ ID NO:1 or a nucleotide sequence encoding another CD28 polypeptide. In another embodiment, a nucleic acid molecule of the present invention comprises a nucleotide sequence which is at least about 10, 20, 50, 100, 200, 400, 450, 500, 550, 600, 650, 700, 750, 800, 900, 1000, 1100, 1200, 1300, 1400, 1500, or more nucleotides in length and hybridizes under stringent hybridization conditions to a nucleic acid molecule of SEQ ID NO:1 or a nucleotide sequence encoding another CD28 polypeptide or the complement thereof.

In another embodiment, a nucleic acid molecule of the invention comprises at least about 10, 20, 50, 100, 200, 400, 450, 500, 550, 600, 650, 700, 750, 800, 900, 1000, 1100, 1200, 1300, 1400, 1500, or more contiguous nucleotides of SEQ ID NO:1 or a nucleic acid molecule encoding another CD28 polypeptide.

In other embodiments, a nucleic acid molecule of the invention has at least 70% identity, more preferably 80% identity, and even more preferably 90% identity with a nucleic acid molecule comprising: at least about 300, 400, 500, 600, 700, 800, 900, 1000, 1100, 1200, 1300, 1400 or about 1500 nucleotides of SEQ ID NO: 1 or a nucleic acid molecule encoding another CD28 polypeptide.

A nucleic acid fragment encoding a “biologically active portion of a CD28 protein” can be prepared by isolating a portion of the nucleotide sequence of SEQ ID NO:1, or a nucleotide sequence encoding another CD28 polypeptide, which encodes a polypeptide having a CD28 biological activity (e.g., the ability to modulate proliferation or apoptosis), expressing the encoded portion of the CD28 protein (e.g., by recombinant expression in vitro) and assessing the activity of the encoded portion of the CD28 protein.

Nucleic acid molecules that differ from SEQ ID NO:1 or a nucleic acid molecule encoding another CD28 polypeptide due to degeneracy of the genetic code, and thus encode the same CD28 protein as that encoded by SEQ ID NO:1 or a nucleic acid molecule encoding another CD28 polypeptide are encompassed by the invention. Accordingly, in another embodiment, an isolated nucleic acid molecule of the invention has a nucleotide sequence encoding a protein having an amino acid sequence shown in SEQ ID NO:2 or an amino acid sequence of another CD28 polypeptide.

In addition to the CD28 nucleotide sequence shown in SEQ ID NO:1 or a nucleotide sequence encoding another CD28 polypeptide, it will be appreciated by those skilled in the art that DNA sequence polymorphisms that lead to changes in the amino acid sequences of the CD28 proteins may exist within a population (e.g., the human population). Such genetic polymorphism in the CD28 genes may exist among individuals within a population due to natural allelic variation. The production of such natural allelic variations include both functional and non-functional CD28 proteins and can typically result in 1-5% variance in the nucleotide sequence of a CD28 gene. Any and all such nucleotide variations and resulting amino acid polymorphisms in CD28 genes that are the result of natural allelic variation and that do not alter one or more flnctional activities of a CD28 protein, or at least preserve one or more known functions of the CD28 protein, may be useful in the claimed methods.

Nucleic acid molecules encoding CD28 proteins from different species, and thus which have a nucleotide sequence which differs from the CD28 sequence of SEQ ID NO:1 or a nucleotide sequence encoding another CD28 polypeptide can be used in the claimed methods. Moreover, nucleic acid molecules encoding other CD28 family members and, thus, which have a nucleotide sequence which differs from the CD28 family sequence of SEQ ID NO:1 or a nucleotide sequence encoding another CD28 polypeptide are intended to be within the scope of the invention.

Nucleic acid molecules corresponding to natural allelic variants and homologues of the CD28 molecules of the invention can be isolated based on their homology to the CD28 nucleic acids disclosed herein using the cDNAs disclosed herein, or portions thereof, as a hybridization probe according to standard hybridization techniques. For example, a CD28 DNA can be isolated from a human genomic DNA library using all or portion of SEQ ID NO:1 or a nucleotide sequence encoding another CD28 polypeptide as a hybridization probe employing standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989). Moreover, a nucleic acid molecule encompassing all or a portion of a CD28 gene can be isolated by the polymerase chain reaction using oligonucleotide primers designed based upon the sequence of SEQ ID NO:1 or a nucleic acid molecule encoding another CD28 polypeptide. For example, mRNA can be isolated from cells (e.g., by the guanidinium-thiocyanate extraction procedure of Chirgwin et al., 1979, Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO:1 or a nucleic acid molecule encoding another CD28 polypeptide. A nucleic acid molecule of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a CD28 nucleotide sequence can be prepared by standard synthetic techniques, e.g., using an automated DNA synthesizer.

In another embodiment, an isolated nucleic acid molecule of the invention can be identified based on shared nucleotide sequence identity using a mathematical algorithm. Such algorithms are outlined in more detail below.

In another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, or a portion thereof, or a nucleotide sequence encoding another CD28 polypeptide, or the respective complements thereof. In other embodiment, the nucleic acid molecule is at least 30, 50, 100, 150, 200, 250, 300, 350, 400, 450, 500, 550, or 600 nucleotides in length. As used herein, the term “hybridizes under stringent conditions” is intended to describe conditions for hybridization and washing under which nucleotide sequences at least 30%, 40%, 50%, or 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the above discussed sequences of SEQ ID NO:1 or a nucleic acid molecule encoding another CD28 polypeptide, or the respective complements, corresponds to a naturally-occurring nucleic acid molecule.

The skilled artisan will further appreciate that minor changes may be introduced by mutation into nucleotide sequences, e.g., of SEQ ID NO:1 or a nucleic acid molecule encoding another CD28 polypeptide, thereby leading to changes in the amino acid sequence of the encoded protein, without altering the functional activity of a CD28 protein. For example, nucleotide substitutions leading to amino acid substitutions at “non-essential” amino acid residues may be made in the sequence of SEQ ID NO:1 or a nucleic acid molecule encoding another CD28 polypeptide. A “non-essential” amino acid residue is a residue that can be altered from the wild-type sequence of a CD28 nucleic acid molecule (e.g., the sequence of SEQ ID NO:1 or a nucleic acid molecule encoding another CD28 polypeptide) without altering the functional activity of a CD28 molecule. Exemplary residues which are non-essential and, therefore, amenable to substitution, can be identified by one of ordinary skill in the art by performing an amino acid alignment of CD28-related molecules and determining residues that are not conserved. Such residues, because they have not been conserved, are more likely amenable to substitution. Non-essential residues can further be confirmed by performing a functional assay on the generated substituted protein product to verify the preservation of function.

Accordingly, another aspect of the invention pertains to nucleic acid molecules encoding CD28 proteins, or above described portions thereof, that contain changes in amino acid residues that are not essential for a CD28 activity. Such CD28 proteins differ in amino acid sequence from SEQ ID NO:2 or an amino acid sequence of another CD28 polypeptide yet retain an inherent CD28 activity. An isolated nucleic acid molecule encoding a non-natural variant of a CD28 protein can be created by introducing one or more nucleotide substitutions, additions or deletions into the nucleotide sequence of SEQ ID NO:1 or a nucleic acid molecule encoding another CD28 polypeptide such that one or more amino acid substitutions, additions or deletions are introduced into the encoded protein. Mutations can be introduced into SEQ ID NO: 1, a fragment thereof, or a nucleic acid molecule encoding another CD28 polypeptide by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, Histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in CD28 is preferably replaced with another amino acid residue from the same side chain family.

Alternatively, in another embodiment, mutations can be introduced randomly along all or part of a CD28 coding sequence, such as by saturation mutagenesis, and the resultant mutants can be screened to identify mutants that retain functional activity. Following mutagenesis, the encoded CD28 mutant protein can be expressed recombinantly in a host cell and the functional activity of the mutant protein can be determined using assays available in the art for assessing a CD28 activity.

Yet another aspect of the invention pertains to isolated nucleic acid molecules encoding a CD28 fusion protein. Such nucleic acid molecules, comprising at least a first nucleotide sequence encoding a full-length CD28 protein, or polypeptide fragment having a CD28 activity, operatively linked to a second nucleotide sequence encoding a non- CD28 protein, can be prepared by standard recombinant DNA techniques.

Isolated CD28 proteins, and biologically active portions thereof can also be used as modulating agents, as well as polypeptide fragments suitable for use as immunogens to raise anti-CD28 antibodies. In one embodiment, native CD28 proteins can be isolated from cells or tissue sources by an appropriate purification scheme using standard protein purification techniques. In another embodiment, CD28 proteins are produced by recombinant DNA techniques. Alternative to recombinant expression, a CD28 protein or polypeptide can be synthesized chemically using standard peptide synthesis techniques. It will be understood that in discussing the uses of CD28 proteins (e.g., as shown in SEQ. ID NO:2 or an amino acid sequence encoding another CD28 polypeptide), that fragments of such proteins that are not full length CD28 polypeptides (e.g, that comprise one or more CD28 domains, e.g. a domain comprising the 16 amino terminal amino acids of SEQ ID NO:2) are included.

Another aspect of the invention pertains to isolated CD28 proteins. Preferably, the CD28 protein comprises the amino acid sequence encoded by SEQ ID NO:1, a portion thereof, or a nucleotide sequence encoding another CD28 polypeptide or a portion thereof. In another preferred embodiment, the protein comprises the amino acid sequence of SEQ ID NO:2, or a portion thereof, or an amino acid sequence of another CD28 polypeptide or a portion thereof. In other embodiments, the protein has at least 50%, at least 60% amino acid identity, more preferably 70% amino acid identity, more preferably 80%, and even more preferably, 90% or 95% amino acid identity with the relevant portion of the amino acid sequence shown in SEQ ID NO:2, or an amino acid sequence of another CD28 polypeptide or a portion thereof, e.g., the consensus domains 20 set forth above. Preferably, the identity to SEQ ID NO:2 spans the entire length of the CD28 protein of the present invention.

Preferred portions of CD28 polypeptide molecules are biologically active, i.e., encode a portion of the CD28 polypeptide having the ability to modulate cell survival and/or proliferation. Preferably, the cell is a T cell, e.g., a Th1 cell.

Biologically active portions of a CD28 protein include peptides comprising amino acid sequences sufficiently homologous to or derived from the amino acid sequence of the CD28 protein, which include less amino acids than the full length CD28 proteins, and exhibit at least one activity of a CD28 protein known in the art or described herein.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which are introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. A non-limiting example of a mathematical algorithm utilized for comparison of sequences is the algorithm of Karlin and Altschul, 1990, Proc. Natl. Acad. Sci. USA 87:2264, modified as in Karlin and Altschul, 1993, Proc. Natl. Acad. Sci. USA 90:5873. Such an algorithm is incorporated into the NBLAST and XBLAST programs (version 2.0) of Altschul, et al., 1990, J. Mol. Biol. 215:403. BLAST nucleotide searches can be performed with the NBLAST program score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., 1997, Nucleic Acids Research 25(17):3389. When utilizing BLAST and Gapped BLAST programs, the default parameters of the respective programs (e.g., XBLAST and NBLAST) can be used. Another preferred, non-limiting algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a mathematical algorithm utilized for the alignment of protein sequences is the Lipman-Pearson algorithm (Lipman and Pearson, 1985, Science 227:1435). When using the Lipman-Pearson algorithm, a PAM250 weight residue table, a gap length penalty of 12, a gap penalty of 4, and a Kutple of 2 can be used. A preferred, non-limiting example of a mathematical algorithm utilized for the alignment of nucleic acid sequences is the Wilbur-Lipman algorithm (Wilbur and Lipman, 1983, Proc. NatL Acad. Sci. USA 80:726). When using the Wilbur-Lipman algorithm, a window of 20, gap penalty of 3, Ktuple of 3 can be used. Both the Lipman-Pearson algorithm and the Wilbur-Lipman algorithm are incorporated, for example, into the MEGALIGN program (e.g., version 3.1.7) which is part of the DNASTAR sequence analysis software package.

Additional algorithms for sequence analysis are known in the art, and include ADVANCE and ADAM., described in Torelli and Robotti, 1994, Comput. Appl. Biosci. 10:3; and FASTA, described in Pearson and Lipman, 1988, Proc. Natl. Acad. Sci USA 85:2444.

In a preferred embodiment, the percent identity between two amino acid sequences is determined using the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6.

Protein alignments can also be made using the Geneworks global protein alignment program (e.g., version 2.5.1) with the cost to open gap set at 5, the cost to lengthen gap set at 5, the minimum diagonal length set at 4, the maximum diagonal offset set at 130, the consensus cutoff set at 50% and utilizing the Pam 250 matrix.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul el al., 1990, J. Mol. Biol. 215:403-10. BLAST nucleotide searches can be performed with the NBLAST program, score=100, wordlength =12 to obtain nucleotide sequences homologous to CD28 nucleic acid molecules of the invention. BLAST protein searches can be performed with the XBLAST program, score=50, wordlength=3 to obtain amino acid sequences homologous to CD28 protein molecules of the invention. To obtain gapped alignments for comparison purposes, Gapped BLAST can be utilized as described in Altschul et al., 1997, Nucleic Acids Res. 25(17):3389-3402. When utilizing BLAST and Gapped BLAST programs, the default parameters of the respective programs (e.g., XBLAST and NBLAST) can be used. For example, the nucleotide sequences of the invention can be analyzed using the default BLASTN matrix 1-3 with gap penalties set at: existence 11 and extension 1. The amino acid sequences of the invention can be analyzed using the default settings: the Blosum62 matrix with gap penalties set at existence 11 and extension 1.

The invention also provides CD28 chimeric or fusion proteins. As used herein, a CD28 “chimeric protein” or “fusion protein” comprises a CD28 polypeptide operatively linked to a non- CD28 polypeptide. A “CD28 polypeptide” refers to a polypeptide having an amino acid sequence corresponding to CD28 polypeptide, or a portion thereof, whereas a “non-CD28 polypeptide” refers to a polypeptide having an amino acid sequence corresponding to a protein which is not substantially homologous to the CD28 protein, e.g., a protein which is different from the CD28 protein and which is derived from the same or a different organism. Within a CD28 fusion protein the CD28 polypeptide can correspond to all or a portion of a CD28 protein. In a preferred embodiment, a CD28 fusion protein comprises at least one biologically active portion of a CD28 protein, e.g., a CD28 consensus domain. Within the fusion protein, the term “operatively linked” is intended to indicate that the CD28 polypeptide and the non-CD28 polypeptide are fused to each other such that they retain their independent functions (e.g., catalytic activity, binding activity, antigenicity, etc.). The non-CD28 polypeptide can be fused to the N-terminus or C-terminus of the CD28 polypeptide. It may be beneficial to include a linker polypeptide as spacer at the fusion junction.

For example, in one embodiment, the fusion protein is a GST-CD28 fusion protein in which the CD28 sequences are fused to the C-terminus of the GST sequences. In another embodiment, the fusion protein is a CD28 -HA fusion protein in which the CD28 amino acids are fused to an influenza haemagglutinin (HA) epitope tag. This can be accomplished by inserting CD28 encoding nucleotide sequence is in a vector such as pCEP4-HA vector (Herrscher, R. F. et al., 1995, Genes Dev. 9:3067-3082) such that the CD28 nucleotide sequences are fused in-frame to nucleotide sequences encoding an influenza haemagglutinin epitope tag. Such fusion proteins can facilitate the purification of a recombinant CD28 member.

Fusion proteins and peptides produced by recombinant techniques may be secreted and isolated from a mixture of cells and medium containing the protein or peptide. Alternatively, the protein or peptide may be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, a CD28 fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A CD28 encoding nucleic acid can be cloned into such an expression vector such that the fusion moiety is linked in-frame to the CD28 protein.

In another embodiment, the fusion protein is a CD28 protein, or portion thereof, containing a heterologous signal sequence at its N-terminus. In certain host cells (e.g., mammalian host cells), expression and/or secretion of CD28 can be increased through use of a heterologous signal sequence. The CD28 fusion proteins of the invention can be incorporated into pharmaceutical compositions and administered to a subject in vivo. Use of CD28 fusion proteins may be useful therapeutically for the treatment of disorders, e.g., as soluble antagonists of the CD28 ligand. Disorders that would benefit from such treatment include, e.g. cancer or Alzheimer's disease. Such Fc fusion proteins can be used as soluble antagonists of the CD28 ligand. Moreover, the CD28-fusion proteins of the invention can be used as immunogens to produce anti- CD28 antibodies in a subject.

In one embodiment, a CD28-Fc fusion protein can be made using techniques that are known in the art. For example, as taught in the instant examples, a soluble CD28-Fc fusion protein can be constructed by joining the cDNA sequence encoding the extracellular region of CD28 to the hinge-C_{H}2-C_{H}3 regions of human immunoglobulin (Ig). Any isotype may be used in making such a construct, for example, Fc γ1, γ2, γ3, or γ4. Cells can be transfected with a plasmid carrying the CD28-Ig construct, cultured, and conditioned medium harvested. The fusion protein can then be purified, e.g., using a column of immobilized protein A.

In another embodiment, allotypic variants of Fc sequences could be used to construct Fc fusion proteins. In another embodiment, mutations which block effector functions, such as, for example, complement and Fe receptor binding (Armour et al., 1999, Eur. J Immunol., 29:2613; Morgan et al., 1995, Immunology 86: 319; Lund et al, 1991. J.Immunol. 147:2657) could be incorporated into the fusion protein.

The present invention also pertains to variants of the CD28 proteins which function as either CD28 agonists (mimetics) or as CD28 antagonists. Variants of the CD28 proteins can be generated by mutagenesis, (e.g., discrete point mutation or truncation of a CD28 protein). An agonist of the CD28 proteins can retain substantially the same, or a subset, of the biological activities (e.g., binding properties) of the naturally occurring form of a CD28 protein. An antagonist of a CD28 protein can inhibit one or more of the activities of the naturally occurring form of the CD28 protein by, for example, competitively modulating a cellular activity of a CD28 protein. Thus, specific biological effects can be elicited by using a variant of limited function. In one embodiment, treatment of a subject with a variant having a subset of the biological activities of the naturally occurring form of the protein has fewer side effects in a subject relative to treatment with the naturally occurring form of the CD28 protein.

In another embodiment, the invention pertains to a derivative of CD28 which is formed by modification of at least one amino acid residue of CD28 by oxidation, reduction, or other derivatization processes known in the art. Such derivatives are also useful in the methods described herein.

Variants of a CD28 protein which function as either CD28 agonists (mimetics) or as CD28 antagonists are identified by screening combinatorial libraries of mutants, (e.g., point mutants or truncation mutants) of a CD28 protein for CD28 protein agonist or antagonist activity. The process of said screening method is also considered an aspect of the present invention. In one embodiment, a variegated library of CD28 variants is encoded by a variegated gene library generated by combinatorial mutagenesis at the nucleic acid level. A variegated library of CD28 variants can be produced by, for example, enzymatically ligating a mixture of synthetic oligonucleotides into gene sequences such that a degenerate set of potential CD28 sequences is expressible as individual polypeptides, or alternatively, as a set of larger fusion proteins (e.g., for phage display) containing the set of CD28 sequences therein. There are a variety of methods which can be used to produce libraries of potential CD28 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CD28 sequences. Methods for synthesizing-degenerate oligonucleotides are known in the art (see, e.g., Narang, S.A., 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

In addition, libraries of fragments of a CD28 protein coding sequence can be used to generate a variegated population of CD28 fragments for screening and subsequent selection of variants of a CD28 protein. The library is generated from a library of coding sequence (nucleic acid) fragments. The library of coding sequence fragments can be generated by treating a double stranded PCR fragment of a CD28 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the CD28 protein.

Several techniques are known in the art for screening protein products of combinatorial libraries made by point mutations or truncation, and for screening cDNA libraries for protein products of gene libraries having a selected property. Such techniques are adaptable for rapid screening of the gene libraries generated by the combinatorial mutagenesis of CD28 proteins. The most widely used techniques, which are amenable to high through-put analysis, for screening large gene libraries typically include cloning a gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify CD28 variants (Arkin and Yourvan, 1992, Proc. Natl. Acad. Sci. USA 89:7811-7815; Delgrave et al., 1993, Protein Engineering 6(3):327-331).

In one embodiment, cell based assays (assays which utilize single cells rather than whole tissues or organisms) are exploited to analyze a variegated CD28 library. For example, a library of expression vectors are transfected into a cell line which ordinarily synthesizes and secretes CD28. The transfected cells are then cultured such that CD28 and a particular mutant CD28 are secreted and the effect of expression of the mutant on CD28 activity in cell supernatants is then assayed, e.g., by any of a number of activity assays. Plasmid DNA is then recovered from the cells which score for inhibition, or alternatively, potentiation of CD28 activity, and the individual clones further characterized.

The present invention also pertains to variants of CD28 proteins which function as either agonists (mimetics) or as antagonists of a CD28 motif. Variants of the CD28 proteins can be generated by mutagenesis, e.g., discrete point mutation or truncation of a CD28 protein. A polypeptide having an agonist activity of a CD28 protein motif can retain substantially the same, or a subset, of the biological activities of the naturally occurring form of the motif, while lacking other activities. An antagonist of a CD28 protein motif can inhibit one or more of the activities of the CD28 motif by, for example, failing to perform that activity. For example, a molecule comprising a CD28 survival motif, while lacking a CD28 proliferation motif, is an agonist of a CD28 survival motif and an antagonist of a CD28 proliferation motif. Alternatively, since the survival motifs are thought to function via recruiting other peptides (e.g. tyrosine kinases) a variant which contains the motif, expressed in the cell at a different location than wild type CD28 (e.g., in the cytoplasm rather than at the plasma membrane) may serve as an antagonist by competitively inhibiting binding of recruited proteins to endogenous motifs. Thus, specific biological effects can be elicited by treatment with a variant of limited function. In one embodiment, variants of a CD28 protein which function as either CD28 agonists (mimetics) or as CD28 antagonists can be identified by screening combinatorial libraries of mutants, e.g., truncation mutants, of a CD28 protein for CD28 protein agonist or antagonist activity.

In addition to CD28 polypeptides consisting only of naturally-occurring amino acids, CD28 peptidomimetics are also encompassed by the present invention. Peptide analogs are commonly used in the pharmaceutical industry as non-peptide drugs with properties analogous to those of the template peptide. These types of non-peptide compound are termed “peptide mimetics” or “peptidomimetics” (Fauchere, J., 1986, Adv. Drug Res. 15: 29; Veber and Freidinger, 1985, TINS p.392; and Evans et al., 1987, J. Med. Chem 30: 1229, which are incorporated herein by reference) and are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as human CD28, but have one or more peptide linkages optionally replaced by a linkage selected from the group consisting of: —CH2NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., 1980, Trends Pharm Sci pp. 463-468 (general review); Hudson, D. et al, 1979, Int J Pept Prot Res 14:177-185 (—CH₂NH—, CH₂CH₂—); Spatola, A. F. et al., 1986, Life Sci 38:1243-1249 (—CH₂—S); Hann, M. M., 1982, J Chem Soc Perkin Trans 1 307-314 (—CH—CH—, cis and trans); Almquist, R. G. et al, 1980, J Med Chem 23:1392-1398 (—COCH₂—); Jennings-White, C. et al., 1982, Tetrahedron Lett 23:2533 (—COCH₂—); Szelke, M. et al., 1982, European Appln. EP 45665 CA: 97:39405 (1982)(—CH(OH)CH₂—); Holladay, M. W. et al., 1983, Tetrahedron Lett .24:4401-4404 (—C(OH)CH₂—); and Hruby, V. J., 1982, Life Sci 31:189-199 (—CH₂—S—); each of which is incorporated herein by reference. A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of a CD28 amino acid sequence with a D-amino acid of the same type (e.g., D-lysine in place of L-lysine) may be used to generate more stable peptides. In addition, constrained peptides comprising a CD28 amino acid sequence or a substantially identical sequence variation may be generated by methods known in the art (Rizo and Gierasch, 1992, Ann. Rev. Biochem. 61: 387, incorporated herein by reference); for example, by adding internal cysteine residues capable of forming intramolecular disulfide bridges which cyclize the peptide.

The amino acid sequences of CD28 polypeptides identified herein will enable those of skill in the art to produce polypeptides corresponding to CD28 peptide sequences and sequence variants thereof. Such polypeptides may be produced in prokaryotic or eukaryotic host cells by expression of polynucleotides encoding a CD28 peptide sequence, frequently as part of a larger polypeptide. Alternatively, such peptides may be synthesized by chemical methods. Methods for expression of heterologous proteins in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume. 152; Guide to Molecular Cloning Techniques (1987), Academic Press, Inc., San Diego, Calif.; Merrifield, J., 1969, J. Am. Chem. Soc. 91: 501; Chaiken I. M. ,1981, CRC Crit. Rev. Biochem. 11: 255, Kaiser et al., 1989, Science 243: 187; Merrifield, B., 1986, Science 232: 342; Kent, S. B. H., 1988, Ann. Rev. Biochem. 57: 957; and Offord, R. E., 1980, Semisynthetic Proteins, Wiley Publishing, which are incorporated herein by reference.

Peptides can also be produced, typically by direct chemical synthesis, and used e.g., as agonists or antagonists of a CD28/CD28 binding protein interaction. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides may be used therapeutically to treat disease, e.g., by altering the process of cell proliferation, differentiation or apoptosis in a cell population of a patient.

An isolated CD28 protein, or a portion or fragment thereof, can also be used as an immunogen to generate antibodies that bind CD28 using standard techniques for polyclonal and monoclonal antibody preparation. A full-length CD28 protein or, alternatively, the antigenic peptide fragments of CD28 can be used as immunogens. The antigenic peptide of CD28 comprises at least 8 amino acid residues and encompasses an epitope of CD28 such that an antibody raised against the peptide forms a specific immune complex with CD28. Preferably, the antigenic peptide comprises at least 10 amino acid residues, more preferably at least 15 amino acid residues, even more preferably at least 20 amino acid residues, and most preferably at least 30 amino acid residues.

Alternatively, an antigenic peptide fragment of a CD28 polypeptide can be used as the immunogen. An antigenic peptide fragment of a CD28 polypeptide typically comprises at least 8 amino acid residues of the amino acid sequence shown in SEQ ID NO:2 or an amino acid sequence of another CD28 polypeptide and encompasses an epitope of a CD28 polypeptide such that an antibody raised against the peptide forms an immune complex with a CD28 molecule. Preferred epitopes encompassed by the antigenic peptide are regions of CD28 that are located on the surface of the protein, e.g., hydrophilic regions. In one embodiment, the antibody generated binds substantially specifically to a full length CD28 molecule.

Preferably, the antigenic peptide comprises at least about 10 amino acid residues, more preferably at least about 15 amino acid residues, even more preferably at least 20 about amino acid residues, and most preferably at least about 30 amino acid residues. Preferred epitopes encompassed by the antigenic peptide are regions of a CD28 polypeptide that are located on the surface of the protein, e.g., hydrophilic regions, and that are unique to a CD28 polypeptide. In one embodiment such epitopes can be specific for a CD28 proteins from one species, such as mouse or human (i.e., an antigenic peptide that spans a region of a CD28 polypeptide that is not conserved across species is used as immunogen; such non conserved residues can be determined using an alignment such as that provided herein). A standard hydrophobicity analysis of the protein can be performed to identify hydrophilic regions.

A CD28 immunogen typically is used to prepare antibodies by immunizing a suitable subject, (e.g., rabbit, goat, mouse or other mammal) with the immunogen. An appropriate immunogenic preparation contains, for example, a recombinantly expressed CD28 protein or a chemically synthesized CD28 peptide. The preparation can further include an adjuvant, such as Freund's complete or incomplete adjuvant, or similar immunostimulatory agent. Immunization of a suitable subject with an immunogenic CD28 preparation induces a polyclonal anti- CD28 antibody response.

Accordingly, another aspect of the invention pertains to anti- CD28 polypeptide antibodies and methods of their use. Such antibodies can be used as agonists and/or antagonists of CD28 polypeptides. In a preferred embodiment antibodies specifically recognize CD28 and not another CD28 polypeptide. Polyclonal anti-CD28 antibodies can be prepared as described above by immunizing a suitable subject with a CD28 immunogen. The anti-CD28 antibody titer in the immunized subject can be monitored over time by standard techniques, such as with an enzyme linked immunosorbent assay (ELISA) using immobilized a CD28 polypeptide. If desired, the antibody molecules directed against a CD28 polypeptide can be isolated from the mammal (e.g., from the blood) and further purified by well known techniques, such as protein A chromatography to obtain the IgG fraction. At an appropriate time after immunization, e.g., when the anti- CD28 antibody titers are highest, antibody-producing cells can be obtained from the subject and used to prepare monoclonal antibodies by standard techniques, such as the hybridoma technique originally described by Kohler and Milstein (Kohler and Milstein, 1975, Nature 256:495-497) (see also, Brown el al., 1981, J. Immunol 127:539-46; Brown et al.,1980, J Biol Chem 255:4980-83; Yeh et al., 1976, Proc. Natl. Acad. Sci USA 76:2927-31; and Yeh et al., 1982, Int. J. Cancer 29:269-75), the more recent human B cell hybridoma technique (Kozbor et al., 1983, Immunol Today 4:72), the EBV-hybridoma technique (Cole et aL.,l 985, Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner, 1981, Yale J Biol. Med., 54:387-402; M. L. Gefter et al., 1977, Somatic Cell GeneL, 3:231-36). Briefly, an immortal cell line (typically a myeloma) is fused to lymphocytes (typically splenocytes) from a mammal immunized with a CD28 immunogen as described above, and the culture supernatants of the resulting hybridoma cells are screened to identify a hybridoma producing a monoclonal antibody that binds specifically to a CD28 polypeptide.

Any of the many well known protocols used for fusing lymphocytes and immortalized cell lines can be applied for the purpose of generating an anti- CD28 monoclonal antibody (see, e.g., G. Galfre et al., 1977, Nature 266:55052; Gefter et al. Somatic Cell Genet., cited supra; Lerner, Yale J Biol. Med., cited supra; Kenneth, Monoclonal Antibodies, cited supra). Moreover, the ordinary skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Agl4 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a CD28 molecule, e.g., using a standard ELISA assay.

As an alternative to preparing monoclonal antibody-secreting hybridomas, a monoclonal anti-CD28 antibody can be identified and isolated by screening a recombinant combinatorial immunoglobulin library (e.g., an antibody phage display library) with a CD28 to thereby isolate immunoglobulin library members that bind a CD28 polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al., 1991. Bio/Technology 9:1370-1372; Hay et al., 1992, Hum Antibod Hybridomas 3:81 -85; Huse et al., 1989, Science 246:1275-1281; Griffiths et al., 1993, EMBO J 12:725-734; Hawkins et aL, 1992, J Mol Biol 226:889-896; Clarkson et al., 1991, Nature 352:624-628; Gram et al., 1992, Proc. Natl. Acad. Sci USA 89:3576-3580; Garrad et al., 1991, Bio/Technology 9:1373-1377; Hoogenboom et al., 1991, Nuc Acid Res 19:4133-4137; Barbas et al., 1991, Proc. Nail Acad. Sci USA 88:7978-7982; and McCafferty et al., 1990, Nature 348:552-554.

Additionally, recombinant anti- CD28 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No.,4,816,567; Cabilly et aL European Patent Application 125,023; Better et al., 1988, Science 240:1041-1043; Liu et al., 1987, Proc. Natl. Acad. Sci USA 84:3439-3443; Liu et al., 1987, J. Immunol. 139:3521-3526; Sun et al., 1987, Proc. Natl. Acad. Sci USA 84:214-218; Nishimura et al., 1987, Canc. Res. 47:999-1005; Wood et al., 1985, Nature 314:446-449; and Shaw et al., 1988, J. Natl Cancer Inst. 80:1553-1559; Morrison, S. L., 1985, Science 229:1202-1207; Oi et al., 1986, BioTechniques 4:214; Winter U.S. Pat. No. 5,225,539; Jones et al., 1986, Nature 321:552-525; Verhoeyan et al., 1988, Science 239:1534; and Beidler et al., 1988, J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743.

An anti- CD28 antibody (e.g., monoclonal antibody) can be used to isolate a CD28 polypeptide by standard techniques, such as affinity chromatography or immunoprecipitation. Anti- CD28 antibodies can facilitate the purification of natural CD28 polypeptides from cells and of recombinantly produced CD28 polypeptides expressed in host cells. Moreover, an anti- CD28 antibody can be used to detect a CD28 protein (e.g., in a cellular lysate or cell supernatant). Detection may be facilitated by coupling (i.e., physically linking) the antibody to a detectable substance. Accordingly, in one embodiment, an anti- CD28 antibody of the invention is labeled with a detectable substance. Examples/of detectable substances include various enzymes, prosthetic groups, fluorescent materials, luminescent materials and radioactive materials. Examples of suitable enzymes include horseradish peroxidase, alkaline phosphatase,β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamnine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

Accordingly, in one embodiment, anti-CD28 antibodies can be used, e.g., intracellularly to inhibit protein activity. The use of intracellular antibodies to inhibit protein function in a cell is known in the art (see e.g., Carlson, J. R., 1988, Mol. Cell. Biol. 8:2638-2646; Biocca, S. et al., 1990, EMBO J. 9:101-108; Werge, T. M. et al., 1990, FEBS Letters 274:193-198; Carlson, J. R., 1993, Proc. Natl. Acad. Sci. USA 90:7427-7428; Marasco, W. A. et al., 1993, Proc. Natl. Acad. Sci. USA 90:7889-7893; Biocca, S. et al., 1994, Bio/Technology 12:396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J Biol. Chem. 269:23931-23936; Beerli, R. R. et al., 1994, Biochem. Biophys. Res. Commun. 204:666-672; Mhashilkar, A. M. et al., 1995, EMBO J. 14:1542-1551; Richardson, J. H. et al., 1995, Proc. Natl. Acad. Sci. USA 92:3137-3141; PCT Publication No. WO 94/02610 by Marasco et al.; and PCT Publication No. WO 95/03832 by Duan et al.).

In one embodiment, a recombinant expression vector is prepared which encodes the antibody chains in a form such that, upon introduction of the vector into a cell, the antibody chains are expressed as a functional antibody in an intracellular compartment of the cell. For inhibition of CD28 activity according to the inhibitory methods of the invention, an intracellular antibody that specifically binds the CD28 protein is expressed in the cytoplasm of the cell. An antibody which specifically binds a CD28 proliferation or survival motif will specifically and selectively block the activity of that motif. To prepare an intracellular antibody expression vector, antibody light and heavy chain cDNAs encoding antibody chains specific for the target protein of interest, e.g., CD28, are isolated, typically from a hybridoma that secretes a monoclonal antibody specific for the CD28 protein. Hybridomas secreting anti-CD28 monoclonal antibodies, or recombinant anti-CD28 monoclonal antibodies, can be prepared as described above.

Once a monoclonal antibody specific for a CD28 protein has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or CDNA library screening. For recombinant antibodies, such as from a phage display library, CDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al., 1991, Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. To allow for cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit CD28 activity in a cell, the expression vector encoding the anti- CD28 intracellular antibody is introduced into the cell by standard transfection methods, as discussed herein.

An antibody or antibody portion of the invention can be derivatized or linked to another functional molecule (e.g., a peptide or polypeptide). Accordingly, the antibodies and antibody portions of the invention are intended to include derivatized and otherwise modified forms of the anti-CD28 antibodies described herein, including, e.g., antibodies conjugated to other molecules (e.g., antibodies or polypeptides which bind to other cell markers). For example, an antibody or antibody portion of the invention can be functionally linked (by chemical coupling, genetic fusion, noncovalent association or otherwise) to one or more other molecular entities, such as another antibody (e.g., to create a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

B. Expression of CD28 Polypeptides or Motifs

Recombinant expression vectors useful for expression of CD28 protein in a cell are known in the art. Typically the CD28 cDNA is first introduced into a recombinant expression vector using standard molecular biology techniques. An CD28 cDNA can be obtained, for example, by amplification using the polymerase chain reaction (PCR) or by screening an appropriate cDNA library. The nucleotide sequences of CD28 cDNAs (e.g., mouse and human) are known in the art and can be used for the design of PCR primers that allow for amplification of a cDNA by standard PCR methods, or for the design of a hybridization probe that can be used to screen a cDNA library using standard hybridization methods. The nucleotide and predicted amino acid sequences of a mammalian CD28 cDNA are disclosed in McCaffrey, P. G. et al. (1993) Science 262:750-754 (see also U.S. Pat. No. 5,656,452 by Rao and U.S. Pat. No. 5,612,455 by Hoey).

Following isolation or amplification of a CD28 cDNA, the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid in a form suitable for expression of the nucleic acid in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

It will be appreciated by those skilled in the art that the design of the expression vector may depend on such factors as the choice of the host cell to be transformed, the level of expression of protein desired, etc. When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions,(see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl Acad. Sci. USA 89:5547-5551; Gossen, M. et al (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the □-fetoprotein promoter (Campes and Tilghman (1989) Genes Dev. 3:537-546).

Vector DNA can be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on a separate vector from that encoding a maf family protein or, more preferably, on the same vector. Cells stably transfected with the introduced nucleic acid can be identified by drug selection (e.g., cells that have incorporated the selectable marker gene will survive, while the other cells die).

III. Agents Which Modulate CD28 Survival Motif Activity Another aspect of the present invention relates to methods for identifying compounds.that modulate cell survival using indicator compositions that include a CD28 survival motif. CD28 has been demonstrated to be a regulator of cell survival, and this activity is distinct and separable from the effects of CD28 on cell proliferation. Accordingly, compounds that specifically modulate the ability of CD28 to induce cell survival can be identified, as described herein, and the effect of a selected test compound on survival relative to proliferation can be evaluated.

Thus, another aspect of the invention pertains to screening assays for identifying compounds that modulate cell survival. Such an assay utilizes an indicator composition comprising a CD28 survival motif. The indicator composition is independently contacted with each member of a library of test compounds. The effect of the test compound on the activity of the CD28 survival motif is then assayed. If the test compound contacted exhibits survival motif modulatory activity, the compound is identified as a compound of interest in that it is a candidate compound for possessing cell survival modulatory activity. The effect of the candidate compound on cell survival can then be assayed to determine if the test compound is a compound that modulates cell survival. The effect of the compound of interest on a CD28 proliferation motif, or on cell proliferation can further be assayed, to determine whether the compound modulates CD28 survival motif activity, or cell survival, relative to CD28 proliferation motif activity or cell proliferation. One would expect that the agent that modulates CD28 survival motif activity would not have a direct modulatory effect on the activity of a CD28 proliferation motif.

The indicator composition can be a cell that expresses the CD28 survival motif, for example, a cell that naturally expressed CD28 (e.g., a T cell) or, more preferably, a cell that has been engineered to express the CD28 protein or portion thereof which contains a CD28 survival motif, by introducing into the cell an expression vector encoding the CD28 protein or a portion or variant thereof.

Alternatively, the indicator composition can be a cell-free composition that includes a CD28 survival motif (e.g., a cell extract from a CD28-expressing cell or a composition that includes purified CD28 protein, either natural CD28 or recombinant CD28). In one embodiment, the indicator composition includes a CD28 survival motif and a target molecule with which CD28 interacts (e.g., grb-2, Gads, or a kinase such as P13K), and the ability of the test compound to modulate the interaction of the CD28 protein with a target molecule is monitored to thereby identify the test compound as a modulator of the activity of the CD28 survival motif.

In preferred embodiments, the indicator composition comprises an indicator cell, wherein the indicator cell comprises a CD28 survival motif and a reporter gene responsive to the CD28 survival motif activity. Preferably, the indicator cell contains a recombinant expression vector encoding the CD28 survival motif, and a vector comprising a CD28-responsive regulatory element operatively linked a reporter gene. The screening method preferably comprises contacting the indicator cell with a test compound, and determining the level of expression of the reporter gene in the indicator cell in the presence of the test compound. The level of expression of the reporter gene in the indicator cell in the presence of the test compound is compared with the level of expression of the reporter gene in the indicator cell in the absence of the test compound, to determine if the test compound modulates CD28 survival motif activity. A test compound which modulates CD28 survival motif activity in this assay is thus identified as a compound that modulates the activity of a CD28 protein.

CD28-responsive elements that can be used in the reporter gene construct are known in the art and include, for example, upstream regulatory regions from genes involved in cell survival such as, BCl-X_{L}. A cell that has been engineered to express the CD28 survival motif can be produced by introducing into the cell an expression vector encoding the CD28 protein or the CD28 survival motif, as set forth above.

In another embodiment, the indicator composition is a cell free composition. CD28 expressed by recombinant methods in a host cell can be isolated from the host cells, or cell culture medium using standard methods for protein purification, for example, by ion-exchange chromatography, gel filtration chromatography, ultrafiltration, electrophoresis, and immunoaffinity purification with antibodies specific for CD28. Alternatively, an extract of CD28-expressing cells can be prepared for use as cell-free composition.

In one embodiment, compounds that specifically modulate CD28 activity are identified based on their ability to modulate the interaction of CD28 with a target molecule to which CD28 binds. The target molecule can be a protein, such as a kinase or phosphatase. Suitable assays are known in the art that allow for the detection of protein-protein interactions (e.g., immunoprecipitations, phosphorylation assays) that allow for the detection of interaction between two proteins or phosphorylation of tyrosine residues. By performing such assays in the presence and absence of test compounds, these assays can be used to identify compounds that modulate (e.g., inhibit or enhance) the activity of a CD28 survival motif.

In one embodiment, the activity of a CD28 survival motif in the presence of the test compound is greater than the activity of a CD28 survival motif in the absence of the test compound, in which case the test compound is identified as a compound that enhances activity of a CD28 survival motif. In another embodiment, the activity of a CD28 survival motif in the presence of the test compound is less than the activity of a CD28 survival motif in the absence of the test compound, in which case the test compound is identified as a compound that inhibits activity of a CD28 survival motif.

In the method for identifying a test compound that modulates an interaction between CD28 protein and a target molecule, the full CD28 protein may be used in the method, or, alternatively, only portions of the CD28 protein may be used. For example, an isolated CD28 cytoplasmic tail can be used.

The degree of interaction between CD28 proteins and the target molecule can be determined, for example, by labeling one of the proteins with a detectable substance (e.g., a radiolabel), isolating the non-labeled protein and quantitating the amount of detectable substance that has become associated with the non-labeled protein. The assay can be used to identify compounds that either stimulate or inhibit the interaction between the CD28 protein (via the survival motif) and a target molecule. A test compound that stimulates the interaction between the CD28 survival motif and a target molecule is identified based upon its ability to increase the degree of interaction between the CD28 survival motif and a target molecule as compared to the degree of interaction in the absence of the test compound, whereas a test compound that inhibits the interaction between the CD28 survival motif and a target molecule is identified based upon its ability to decrease the degree of interaction between the CD28 survival motif and a target molecule as compared to the degree of interaction in the absence of the compound. Assay systems for identifying compounds that modulate SH2 domain-ligand interactions as described in U.S. Pat. No. 5,352,660 by Pawson, can be adapted to identify test compounds that modulate CD28 survival motif—target molecule interaction.

Recombinant expression vectors that can be used for expression of CD28, or the portion thereof, in the indicator cell are known in the art (see discussions above). In one embodiment, within the expression vector the CD28-coding sequences are operatively linked to regulatory sequences that allow for constitutive expression of CD28 in the indicator cell (e.g., viral regulatory sequences, such as a cytomegalovirus promoter/enhancer, can be used). Use of a recombinant expression vector that allows for constitutive expression of CD28 in the indicator cell is preferred for identification of compounds that enhance or inhibit the activity of CD28. In an alternative embodiment, within the expression vector the CD28 coding sequences are operatively linked to regulatory sequences of the endogenous CD28 gene (i.e., the promoter regulatory region derived from the endogenous gene). Use of a recombinant expression vector in which CD28 expression is controlled by the endogenous regulatory sequences is preferred for identification of compounds that enhance or inhibit the transcriptional expression of CD28.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

A variety of cell types are suitable for use as an indicator cell in the screening assay. In one embodiment, the cell does not express wild-type CD28. In one embodiment, the cell is a non-T cell. In one embodiment, the cell is a T cell. Preferably, a primary T cell line is used.

In one embodiment, the level of expression of the reporter gene in the indicator cell in the presence of the test compound is higher than the level of expression of the reporter gene in the indicator cell in the absence of the test compound and the test compound is identified as a compound that stimulates the activity of a CD28 survival motif. In another embodiment, the level of expression of the reporter gene in the indicator cell in the presence of the test compound is lower than the level of expression of the reporter gene in the indicator cell in the absence of the test compound and the test compound is identified as a compound that inhibits the expression or activity of a CD28 survival motif.

Alternative to the use of a reporter gene construct, compounds that modulate the expression or activity of a CD28 survival motif can be identified by using other “read-outs.” For example, an indicator cell can be transfected with a CD28 expression vector, incubated in the presence and in the absence of a test compound, and expression of genes associated with cell survival (e.g., BCl-X_{L}) can be measured. Standard methods for detecting gene expression, such as reverse transcription-polymerase chain reaction (RT-PCR) are known in the art.

In another embodiment, the ability of a compound to modulate a CD28 survival motif can be determined by measuring the ability of a compound to modulate apoptosis in a cell. The hallmark of apoptosis is degradation of DNA. Early in the process, this degradation occurs in internucleosomal DNA linker regions. The DNA cleavage may yield double-stranded and single-stranded DNA breaks. Apoptosis can be measured in cells using standard techniques. For example, degradation of genomic DNA of a population of cells can be analyzed by agarose gel electrophoresis, or DNA fragmentation assays based on hu 3H-thymidine or 5-Bromo-2′-deoxy-uridine, can be used.

To analyze apoptosis in individual cells, apoptotic cells may be recognized microscopically because of the characteristic appearance of nuclear chromatin condensation and fragmentation. Apoptosis can be measured in individual cells, for example, using Hoechst stain and looking for cells with pyknotic nuclei as described in the appended Examples. Alternatively, double and single-stranded DNA breaks can be detected by labeling the free 3′-OH termini with modified nucleotides (e.g., biotin-dUTP, DIG-dUTP, fluorescein-dUTP) in an enzymatic reaction. Terminal deoxynucleotidyl transferase (TdT) catalyzes the template independent polymerization of deoxyribonucleotides to the 3′end of the DNA. This method is referred to as TUNEL (TdT-mediated dUTP-X nick end labeling). Alternatively, free 3′OH groups may be labeled using DNA polymerases by nick translation. tunnel staining can be used to identify cells with double stranded DNA breaks. Labeled free 3′OH groups that have incorporated labeled dUTP can be visualized by flow cytometry and/or fluorescence microscopy. Reagents for performing these assays are available e.g., from Roche Molecular Biochemicals USA (In situ cell death detection kit). In addition, annexin (e.g., Annexin-V-Alexa™ 568 commercially available from Roch molecular Biochemicals USA) can be used for this purpose. One of the early plasma membrane changes associated with cells undergoing apoptosis is the translocation of phosphatidylserine from the inner leaflet of the plasma membrane to the outer layer, thereby exposing phosphatidylserine at the surface of the cell. Annexin-V is a phospholipid binding protein which binds to phosphatidyl serine and can be used as a probe for phosphatidylserine on cell surfaces. Annexin-V can be used in combination with a DNA stain (e.g., BOBO™-b 1) to differentiate apoptotic cells from necrotic cells.

In one embodiment, an indicator cell is contacted with a stimulus that is known to modulate T cell survival prior to contacting the cell with a test compound. For example, such stimuli as, UV irradiation, T cell receptor crosslinking, Fas crosslinking, and growth factor deprivation.

Once a test compound is identified that modulates cell survival by modulating a CD28 survival motif, by one of the variety of methods described herein, the selected test compound (or “compound of interest”) can then be further evaluated for its effect on cell proliferation, for example by contacting the compound of interest with a CD28 proliferation motif and determining the effect of the compound of interest on the activity of the CD28 proliferation motif, as compared to an appropriate control (such as untreated cells or cells treated with a control compound, or carrier, that does not modulate cell proliferation). The effect of the test compound on the proliferation of the cells can be determined as described in more detail below (e.g., by analysis of the proliferative capacity of T cells exposed to the test compound).

A variety of test compounds can be evaluated using the screening assays described herein. In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303- ), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061- ).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al. (1996) Immunopharmacology 33:68- ; and in Gallop et al. (1994); J. Med. Chem. 37:1233-.

Libraries of compounds may be presented in solution (e.g., Houghten (1992) Biotechniques 13:412-421), or affixed to a solid or semisolid support, e.g., on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); In still another embodiment, the combinatorial polypeptides are produced from a cDNA library.

Exemplary compounds which can be screened for activity include, but are not limited to, peptides, nucleic acids, carbohydrates, small organic molecules, and natural product extract libraries.

In addition to the identification of novel agents that modulate a CD28 survival motif, known agents can be tested and used for their ability to modulate such a motif. Examples of such agents are set forth below.

A. Inhibitory Compounds

Since inhibition of the activity of a CD28 survival motif is associated with reduced T cell survival, to reduce T cell survival, T cells are contacted with an agent that inhibits CD28 survival motif activity. Cells may be contacted with the agent in vitro. The cells may then be administered to a subject. Alternatively, the agent may be directly administered to a subject by methods which produce contact of the agent with T cells of the subject. The methods of the invention using agents that inhibit a CD28 survival motif can be used in the treatment of disorders in which T cell survival is excessive.

Inhibitory compounds of the invention can be, for example, molecules that act intracellularly to specifically inhibit the activity of a CD28 survival motif. Examples of such molecules include peptidic compounds that inhibit the interaction of a CD28 survival motif with a target molecule (e.g., P13K, grb-2, or gads) or that promote the interaction of a CD28 survival domain with a phosphatase. In one embodiment, the inhibitory agent is a dominant negative molecule. One type of dominant negative is a dominant negative version of the target molecule (e.g., P13K, grb-2, or gads). In signal transduction, a dominant negative molecule generally retains partial activity such that it retains the ability to bind an upstream molecule, but fails to transmit a downstream signal, such that it can inhibit wild type molecules from binding. Often, dominant negative mutants have a higher affinity or much slower off rate than the wild type.

In another embodiment, an agent that inhibits the activity of a CD28 survival motif is an intracellular antibody that binds to (or that affects the activity of) the CD28 survival motif. To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. CD28 survival motifs are found at the plasma membrane, therefore it is preferable that the antibody be expressed such that it has access to the plasma membrane of the cell.

For inhibition of transcription factor activity according to the inhibitory methods of the invention, an intracellular antibody that specifically binds the transcription factor is preferably expressed within the nucleus of the cell. Nuclear expression of an intracellular antibody can be accomplished by removing from the antibody light and heavy chain genes those nucleotide sequences that encode the N-terminal hydrophobic leader sequences and adding nucleotide sequences encoding a nuclear localization signal at either the N- or C-terminus of the light and heavy chain genes (see e.g., Biocca, S. et al. (1990) EMBO J. 9:101-108; Mhashilkar, A. M. et al. (1995) EMBO J. 14:1542-1551). A preferred nuclear localization signal to be used for nuclear targeting of the intracellular antibody chains is the nuclear localization signal of SV40 Large T antigen (see Biocca, S. et al. (1990) EMBO J. 9:101-108; Mhashilkar, A. M. et al. (1995) EMBO J. 14:1542-1551).

Other exemplary inhibitory compounds include peptidic variants of a CD28 molecule, e.g., lacking a CD28 survival domain or comprising an amino acid sequence that inhibits the phosphorylation of tyrosine at position 170 of the wild-type CD28 molecule. The peptidic compounds of the invention can be made intracellularly in cartilage cells by introducing into the cartilage cells an expression vector encoding the peptide. Such expression vectors can be made by standard techniques. The peptide can be expressed intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like). Recombinant methods of making CD28 inhibitory peptides, and methods using them to inhibit CD28 survival motif activity in cells, are described further in Avramburu et al., (1998) Mol Cell. 1:627-637.

Other exemplary inhibitory agents that can be used to specifically inhibit the activity of a CD28 survival motif are chemical compounds that directly inhibit CD28 survival motif activity or inhibit the interaction between CD28 and target molecules. Such compounds can be identified using screening assays that select for such compounds, as described in detail above.

B. Stimulatory Compounds

Since upregulation of the activity of a CD28 survival motif is associated with increased survival, a compound that stimulates the activity of a CD28 survival motif can be used to promote T cell survival. The compound may be contacted to the T cell in vitro or in vivo. In vitro use of the stimulatory compound is useful for prolonging the survival of cells grown in culture for therapeutic use. Such cells are for instance used to produce therapeutic compound. In vitro use of the stimulatory compound may also be used to promote survival of tissues in culture, e.g., of harvested organs which are to be transplanted into a recipient subject. In addition, the T cells contacted in vitro, and then reintroduced into a subject for ex vivo therapy. Contact of the stimulatory compound to the T cell may also be in vivo. As such, CD28 survival motif stimulatory compounds can be used in the treatment of disorders in which T cell survival is aberrantly reduced or in which enhanced T cell survival would be of benefit. For example, HIV infection, immunosuppression, infection, and the like.

Examples of stimulatory compounds include polypeptides comprising a CD28 survival motif, expression vectors encoding a CD28 survival motif and chemical agents that specifically stimulate CD28 survival motif activity.

IV. Agents That Modulate CD28 Proliferation Motif Activity

Another aspect of the invention relates to a method for identifying compounds that modulate cell proliferation using indicator compositions that include a CD28 proliferation motif. CD28 has been demonstrated to be a regulator of cell proliferation, and this activity is distinct and separable from the effects of CD28 on cell survival. Accordingly, compounds that specifically modulate the ability of CD28 to induce cell proliferation can be identified, as described herein, and the effect of a selected test compound on proliferation relative to survival can be evaluated.

Thus, one aspect of the invention pertains to screening assays for identifying compounds that modulate cell proliferation. Such an assay utilizes an indicator composition comprising a CD28 proliferation motif. The indicator composition is independently contacted with each member of a library of test compounds, and the effect (as to proliferation motif activity) of each test compound on the indicator composition is assayed. With the identification of an effect on the proliferation motif activity (increase or decrease), a compound of interest that modulates the activity of a CD28 proliferation motif is selected as a candidate for possessing cell proliferation modulatory activity. The effect of the compound of interest on cell proliferation can then be assayed to determine if the test compound modulates cell proliferation. The effect of the compound of interest on a CD28 survival motif, or on cell survival can further be assayed, to determine whether the compound modulates CD28 proliferation motif activity, or cell proliferation, relative to CD28,survival motif activity or cell survival. One would expect that the agent that modulates CD28 proliferation motif activity would not have a direct modulatory effect on the activity of a CD28 survival motif.

The indicator composition can be a cell that expresses the CD28 proliferation motif, for example, a cell that naturally expressed CD28 (e.g., a T cell) or, more preferably, a cell that has been engineered to express the CD28 protein or portion thereof which contains a CD28 proliferation motif, by introducing into the cell an expression vector encoding the CD28 protein or a portion or variant thereof.

Alternatively, the indicator composition can be a cell-free composition that includes a CD28 proliferation motif (e.g., a cell extract from a CD28-expressing cell or a composition that includes purified CD28 protein, either natural CD28 or recombinant CD28). In one embodiment, the indicator composition includes a CD28 proliferation motif and a target molecule with which CD28 interacts (e.g., an SH3 domain containing protein such as Lck), and the ability of the test compound to modulate the interaction of the CD28 protein with a target molecule is monitored to thereby identify the test compound as a modulator of the activity of the CD28 proliferation motif.

In preferred embodiments, the indicator composition comprises an indicator cell, wherein the indicator cell comprises a CD28 proliferation motif and a reporter gene responsive to the activity of the CD28 proliferation motif. Preferably, the indicator cell contains a recombinant expression vector encoding the CD28 proliferation motif, and a vector comprising a CD28-responsive regulatory element operatively linked a reporter gene. The screening method preferably comprises contacting the indicator cell with a test compound, and determining the level of expression of the reporter gene in the indicator cell in the presence of the test compound. The level of expression of the reporter gene in the indicator cell in the presence of the test compound is compared with the level of expression of the reporter gene in the indicator cell in the absence of the test compound to thereby select a compound, to determine if the test compound modulates CD28 proliferation motif activity. If so, then the test compound is identified as a compound of interest that modulates the activity of CD28 proliferation motif.

CD28-responsive elements that can be used in the reporter gene construct are known in the art and include, for example, upstream regulatory regions from genes involved in cell proliferation such as, cytokine genes, e.g., IL-2. A cell that has been engineered to express the CD28 proliferation motif can be produced by introducing into the cell an expression vector encoding the CD28 protein or the CD28 proliferation motif, as set forth above.

In another embodiment, the indicator composition is a cell free composition. CD28 expressed by recombinant methods in a host cell can be isolated from the host cells, or cell culture medium using standard methods for protein purification, for example, by ion-exchange chromatography, gel filtration chromatography, ultrafiltration, electrophoresis, and immunoaffinity purification with antibodies specific for CD28. Alternatively, an extract of CD28-expressing cells can be prepared for use as cell-free composition.

In one embodiment, compounds that specifically modulate CD28 activity are identified based on their ability to modulate the interaction of CD28 with a target molecule to which CD28 binds. The target molecule can be a protein, such as an SH3 domain, e.g., an SH3 domain of Lck. Suitable assays are known in the art that allow for the detection of protein-protein interactions (e.g., immunoprecipitations) that allow for the detection of interaction between two proteins. By performing such assays in the presence and absence of test compounds, these assays can be used to identify compounds that modulate (e.g., inhibit or enhance) the activity of a CD28 proliferation motif.

In one embodiment, the activity of a CD28 proliferation motif in the presence of the test compound is greater than the activity of a CD28 proliferation motif in the absence of the test compound, in which case the test compound is identified as a compound that enhances activity of a CD28 proliferation motif. In another embodiment, the activity of a CD28 proliferation motif in the presence of the test compound is less than the activity of a CD28 proliferation motif in the absence of the test compound, in which case the test compound is identified as a compound that inhibits activity of a CD28 proliferation motif.

In the method for identifying a compound that modulates an interaction between CD28 protein and a target molecule, the full CD28 protein may be used in the method, or, alternatively, only portions of the CD28 protein may be used. For example, an isolated CD28 cytoplasmic tail can be used.

The degree of interaction between CD28 proteins and the target molecule can be determined, for example, by labeling one -of the proteins with a detectable substance (e.g., a radiolabel), isolating the non-labeled protein and quantitating the amount of detectable substance that has become associated with the non-labeled protein. The assay can be used to identify compounds that either stimulate or inhibit the interaction between the CD28 protein (via the proliferation motif) and a target molecule. A test compound that stimulates the interaction between the CD28 proliferation motif and a target molecule is identified based upon its ability to increase the degree of interaction between the CD28 proliferation motif and a target molecule as compared to the degree of interaction in the absence of the test compound, whereas a test compound that inhibits the interaction between the CD28 proliferation motif and a target molecule is identified based upon its ability to decrease the degree of interaction between the CD28 proliferation motif and a target molecule as compared to the degree of interaction in the absence of the compound. Assay systems for identifying compounds that modulate SH2 domain-ligand interactions as described in U.S. Pat. No. 5,352,660 by Pawson, can be adapted to identify test compounds that modulate CD28 proliferation motif- target molecule interaction.

Recombinant expression vectors that can be used for expression of CD28 or a portion thereof, in the indicator cell are known in the art (see discussions above). In one embodiment, within the expression vector the CD28-coding sequences are operatively linked to regulatory sequences that allow for constitutive expression of CD28 in the indicator cell (eg., viral regulatory sequences, such as a cytomegalovirus promoter/enhancer, can be used). Use of a recombinant expression vector that allows for constitutive expression of CD28 in the indicator cell is preferred for identification of compounds that enhance or inhibit the activity of CD28. In an alternative embodiment, within the expression vector the CD28 coding sequences are operatively linked to regulatory sequences of the endogenous CD28 gene (i.e., the promoter regulatory region derived from the endogenous gene).

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard-methods for measuring the activity of these gene products are known in the art.

A variety of cell types are suitable for use as an indicator cell in the screening assay. In one embodiment, the cell is a non-T cell, in another embodiment, the cell is a T cell. I one embodiment, the cell does not express endogenous CD28. Preferably a primary T cell line is used.

In one embodiment, the level of expression of the reporter gene in the indicator cell in the presence of the test compound is higher than the level of expression of the reporter gene in the indicator cell in the absence of the test compound and the test compound is identified as a compound that stimulates the expression or activity of a CD28 proliferation motif. In another embodiment, the level of expression of the reporter gene in the indicator cell in the presence of the test compound is lower than the level of expression of the reporter gene in the indicator cell in the absence of the test compound and the test compound is identified as a compound that inhibits the expression or activity of a CD28 proliferation motif.

Alternative to the use of a reporter gene construct, compounds that modulate the expression or activity of a CD28 proliferation motif can be identified by using other “read-outs.” For example, an indicator cell can be transfected with a CD28 expression vector, incubated in the presence and in the absence of a test compound, and expression of genes associated with cell proliferation can be measured, e.g., IL-2. Standard methods for detecting gene expression, such as reverse transcription-polymerase chain reaction (RT-PCR) are known in the art.

In another embodiment, the ability of a compound to modulate cell proliferation is measured. Cell proliferation (expansion) can be measured using any of a variety of techniques known in the art, e.g., cells can be enumerated using a hemocytometer or using FACs analysis. Alternatively, cells can be labeled with a radioactive label that is take up by dividing cells (e.g., tritiated thymidine). The degree of proliferation can be determined by assaying the number of counts in the DNA of cells using standard methods.

Other cellular responses can also be measured to determine the effect of a compound on the activity of a CD28 proliferation motif. The translocation of CD28-Lck or the activity of Lck can be measured. In yet another embodiment, IL-2 production can be measured. For example by monitoring IL-2 gene expression or the presence of IL-2 in a cell culture supernatant (e.g., by ELISA or using a bioassay).

In one embodiment, an indicator cell is contacted with a stimulus that is known to transmit a primary activation signal to a T cell prior to contacting the cell with a test compound. For example, such stimuli as T cell receptor crosslinking (e.g., by antibodies specific for the T cell receptor or associated molecules) or by contacting the T cell with antigen in the context of MHC molecules or allogeneic cells, can be used.

Once a test compound is identified that modulates cell proliferation by modulating a CD28 proliferation motif, by one of the variety of methods described herein, the selected test compound (or “compound of interest”) can then be further evaluated for its effect on cell survival. For example the compound of interest can be contacted to a CD28 survival motif and the effect of the compound of interest on the survival of cells determined, as compared to an appropriate control (such as untreated cells or cells treated with a control compound, or carrier, that does not modulate cell proliferation). The effect of the test compound on the proliferation of the cells can be determined as described in more detail below (e.g., by analysis of the proliferative capacity of the cells). Test compounds for use in these screening assays can be obtained by methods described above under the heading of “Agents which Modulate CD28 Survival Motif Activity.”

In addition to the identification of novel agents that modulate a CD28 survival motif, known agents can be tested and used for their ability to modulate such a motif. Exemplary agents are set forth below.

A. Inhibitory Compounds

Since inhibition of the activity of a CD28 proliferation motif is associated with reduced T cell proliferation, to reduce T cell proliferation, T cells are contacted with an agent that innibits a CD28 proliferation motif activity. T cells may be contacted with the agent in vitro. The T cells may then be further used to in vitro for instance in the production of agents which are induced by the inhibition of the proliferation motif. Such agents may be used therapeutically. Alternatively, the in vitro contacted T cell can then be administered to a subject as an ex vivo type therapeutic. Alternatively, the agent may be administered directly to a subject by methods which produce contact of the agent with T cells of the subject. The methods of the invention using agents that inhibit a CD28 proliferation motif can be used in the treatment of disorders in which T cell proliferation is excessive.

Inhibitory compounds of the invention can be, for example, molecules that act intracellularly to specifically inhibit the activity of a CD28 proliferation motif. Examples of such molecules include peptidic compounds that inhibit the interaction of a CD28 proliferation motif with a target molecule (e.g., Lck). In one embodiment, the agent is a dominant negative molecule. One type of dominant negative molecule is a dominant negative form of the target molecule (e.g., Lck).

In another embodiment, an agent that inhibits CD28 proliferation motif activity is an intracellular antibody that binds to (or that affects the activity of) a CD28 proliferation motif. Details regarding the preparation of intracellular antibodies are set forth supra. To make an antibody against a CD28 proliferation motif, polypeptides comprising a CD28 proliferation motif can be used. Other exemplary inhibitory compounds are peptidic variants of a CD28 molecule, e.g., lacking a CD28 proliferation motif of the wild-type CD28 molecule or comprising an amino acid sequence that inhibits the interaction of proline at position 187 and proline at position 190 with an SH3 domain of a heterologous protein (e.g., of Lck). The peptidic compounds of the invention can be made intracellularly in cartilage cells by introducing into the cartilage cells an expression vector encoding the peptide. Such expression vectors can be made by standard techniques. The peptide can be expressed intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like). Recombinant methods of making CD28 inhibitory peptides, and using them to inhibit CD28 proliferation motif activity in cells, are described further in Avramburu et al., (1998) Mol. Cell. 1:627-637.

Other exemplary inhibitory agents for use in specifically inhibiting the activity of a CD28 proliferation motif are chemical compounds that directly inhibit CD28 proliferation motif activity or inhibit the interaction between CD28 and target molecules. Such compounds can be identified using screening assays that select for such compounds, as described in detail above.

B. Stimulatory Compounds

Since upregulation of the activity of a CD28 proliferation motif is associated with increased proliferation, a compound that stimulates the activity of a CD28 proliferation motif can be used to promote T cell proliferation. As such, CD28 proliferation motif stimulatory compounds can be used in vitro or in vivo. In vitro use is for instance to promote the proliferation of T cells in culture (e.g., T cells used in the production of therapeutic agents). These T cells may then be used to produce therapeutic agents or readministered into a subject as an ex vivo type therapeutic. In vivo use is for instance in the treatment of disorders in which T cell proliferation is aberrantly reduced or in which enhanced T cell proliferation would be of benefit. For example, HIV infection, immunosuppression, infection, and the like.

Examples of stimulatory compounds include polypeptides comprising a CD28 proliferation motif, expression vectors encoding a CD28 proliferation motif and chemical agents that specifically stimulate CD28 proliferation motif activity.

For stimulatory or inhibitory agents that comprise nucleic acids (e.g., recombinant expression vectors encoding CD28 motifs, intracellular antibodies or CD28-derived peptides), the compounds can be introduced into cells of a subject using methods known in the art for introducing nucleic acid (e.g., DNA) into cells in vivo. Examples of such methods are set forth above.

V. Methods for Modulating T cell Survival or Proliferation to Modulate an Immune Response

The present invention provides for both prophylactic and therapeutic methods of treating a subject at risk of (or susceptible to) a disorder or having a disorder associated with aberrant cell proliferation or survival. For example, an immune system disorder or condition associated with an undesirable immune response (such as an autoimmune disorder, graft-versus-host disease (GVHD), an allogeneic transplant) or an immune system disorder or condition that would benefit from an enhanced immune response, eg. an immunosuppressed individual.

As used herein, the term “subject” is intended to include living organisms in which an immune response can be elicited. Preferred subjects are mammals. Examples of subjects include humans, monkeys, dogs, cats, mice, rats cows, horses, goats, and sheep. Modulation of CD28 motif activity, therefore, provides a means to regulate proliferation and/or survival in various diseasc states.

A. Prophylactic Methods

In one aspect, the invention provides a method for preventing in a subject, a disease or condition associated with an aberrant or unwanted immune response or, alternatively, an abnormally low immune response, by administering to the subject an agent which downmodulates the activity of a CD28 survival or proliferation motif. Subjects at risk for such disorders can be identified, for example, by any or a combination of diagnostic or prognostic assays known in the art. Administration of a prophylactic agent can occur prior to the manifestation of symptoms characteristic of the aberrant immune response, such that a disease or disorder is prevented or, alternatively, delayed in its progression. Depending on the type of immune response aberrancy, for example, a CD28 proliferation motif modulating or a CD28 survival motif modulating agent can be used for treating a subject. The appropriate agent can be determined based on screening assays described herein.

B. Therapeutic Methods

Another aspect of the invention pertains to methods of modulating CD28 proliferation and/or cell survival regulatory activity for therapeutic purposes. Because the CD28 proliferation motif upregulates cell proliferation and the CD28 survival motif upregulates cell survival, enhanced CD28 survival or proliferation motif activity results in upregulation of immune responses, whereas inhibition of CD28 survival or proliferation motif activity results in downregulation of immune responses.

Modulatory methods of the invention involve contacting a cell (e.g., a T cell) with an agent that modulates the activity of a CD28 motif. An agent that modulates the activity of such a CD28 motif can be an agent as described herein or can be identified using the screening methods described herein.

The modulatory methods of the invention can be performed in vitro (e.g., by contacting the cell with the agent) or, alternatively, in vivo (e.g., by administering the agent to a subject). As such, the present invention provides methods of treating an individual afflicted with a condition or disorder that would benefit from up- or down-modulation of a CD28 motif, e.g., a disorder characterized by an unwanted, insufficient, or otherwise aberrant immune response. In one embodiment, the method involves administering an agent (e.g., an agent identified by a screening assay described herein), or combination of agents that modulates (e.g., upregulates or downregulates) the CD28 motif activity. Preferably, treatment is with either an agent that modulates the CD28 proliferation motif or with an agent that modulates the CD28 survival motif, and not with a combination of agents which together modulate both motifs.

Inhibition of CD28 motif (proliferation or survival) activity is desirable in situations where the immune response is upregulated, for example, when decreased T cell survival or T cell proliferation is likely to have a beneficial effect, for example in a situation of an excessive or unwanted immune response. Such situations include conditions, disorders, or diseases such as an autoimmune disorder (e.g., rheumatoid arthritis, myasthenia gravis, autoimmune thyroiditis, systemic lupus erythematosus, type I diabetes mellitus, Grave's disease, or multiple sclerosis), a transplant (e.g., a bone marrow transplant, a stem cell transplant, a heart transplant, a lung transplant, a liver transplant, a kidney transplant, a cornea transplant, or a skin transplant), graft versus host disease (GVHD), an allergy, or in inflammatory disorder. Treatment of such conditions may optionally involve administration of a second agent, (e.g., a known immunodepressant) to the patient in conjunction with a motif modulatory agent of the present invention.

Likewise, upregulation of CD28 motif activity is desirable in situations in which CD28 is abnormnally downregulated and/or in which increased CD28 motif activity is likely to have a beneficial effect, e.g., in the case of immunosuppressed or tumor-bearing subjects. Treatment of such conditions may optionally involve administration of a second agent (e.g., a known immunostimulant) to the patient in conjunction with a motif modulatory agent of the present invention.

In one embodiment, CD28 proliferation motif activity is stimulated. In another embodiment, CD28 survival motif activity is stimulated.

Identification of compounds that modulate the proliferation or survival of cells by modulating CD28 motifs allows for selective manipulation of cells in a variety of clinical situations using the modulatory methods of the invention.

The stimulation of phosphorylation on tyrosine 170 of a survival motif enhances cell survival, whereas the inhibition of phosphorylation on tyrosine 170 of a survival motif inhibits cell survival. The activity of the survival motif is thought mediated by binding of SH2 domain containing proteins to the CD28 protein which is phosphorylated at tyrosine 170. One such protein is PI-3 kinase (Pages et al., Nature 369: 327-329 (1994)). The stimulation of the interaction between the proline residue at position 187 and the proline residue at position 190 with an interactor molecule (e.g., the SH3 domain of Lck) promotes proliferation and IL-2 production, whereas the inhibition of the interaction between the proline residue at position 187 and the proline residue at position 190 with the interacting molecule reduces proliferation and IL-2 production.

Accordingly, stimulatory methods of the invention (i.e., methods that use an agent which stimulates the motif) result in increased survival or proliferation of T cells. In contrast, the inhibitory methods of the invention (i.e., methods that use an agent which inhibits the motif) inhibit survival or proliferation. Application of the modulatory methods of the invention to the treatment of a disorder may result in cure of the disorder, a decrease in the type or number of symptoms associated with the disorder, either in the long term or short term (i.e., amelioration of the condition) or simply a transient beneficial effect to the subject.

Numerous disorders associated with aberrant cell survival or proliferation have been identified and could benefit from modulation of a CD28 survival or proliferation motif in the individual suffering from the disorder. Application of the immunomodulatory methods of the invention to such disorders is described in further detail below.

VI. Pharmaceutical Compositions

The CD28 motif modulating agents of the invention or CD28 motif modulating agents identified in screening assays of the invention (e.g., nucleic acid molecules, CD28 proteins or portions thereof, modulators of CD28 motif activity) also referred to herein as “active” compounds or substances) can be incorporated into pharmaceutical compositions suitable for administration to a subject, e.g., a human. Such compositions typically comprise the nucleic acid molecule, protein, modulator, or antibody and a pharmaceutically acceptable carrier.

As used herein the language “pharmaceutically acceptable carrier” refers to any and all solvents, dispersion media, coatings, antibacterial and antifungal agents, isotonic and absorption delaying agents, and the like, compatible with pharmaceutical administration. The use of such media and agents for preparation of pharmaceutically active substances is well known in the art. The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. Preferably, it is stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a small molecule, nucleic acid molecule, or peptide) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Without limitation, some examples of suitable carriers in solid dosage form are excipients, and diluents include lactose, dextrose, sucrose, sorbitol, mannitol, starches, gum acacia, calcium phosphate, alginates, calcium silicate, microcrystalline cellulose, olyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring-agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals.

VII. Delivery of Nucleic Acids to Cells

For stimulatory or inhibitory agents that comprise nucleic acids (e.g., recombinant expression vectors encoding CD28 motifs, intracellular antibodies or CD28-derived peptides), the compounds can be introduced into cells of a subject using methods known in the art for introducing nucleic acid (e.g., DNA) into cells in vivo and in vitro. Examples of such methods are described herein.

Direct Injection: Naked DNA can be introduced into cells in vivo by directly injecting the DNA into the cells (see e.g., Acsadi et.al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer especially for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ectopic and amphotrophic retroviral systems include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al (1988) Proc. Natl Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al.(1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g, Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the gene of interest comprised in the nucleic acid molecule can be under control of, for example, the E1A promoter, the major late promoter (MLP) and associated leader sequences, the E3 promoter, or exogenously added promoter sequences.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into T cells. A variety of nucleic acids have been introduced into different cell types using AAV vectors (see for example Hermonat et al. (1984) Proc. Natl. Acad. .Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a ceil can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

VIII. Gene Therapy

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Viral vectors include, for example, recombinant retroviruses, adenovirus, adeno-associated virus, and herpes simplex virus-1. Retrovirus vectors and adeno-associated virus vectors are generally understood to be the recombinant gene delivery system of choice for the transfer of exogenous genes in vivo, particularly into humans. Alternatively they can be used for introducing exogenous genes ex vivo into T cells in culture. These vectors provide efficient delivery of genes into T cells, and the transferred nucleic acids are stably integrated into the chromosomal DNA of the host cell.

A major prerequisite for the use of viruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) is replaced by a gene of interest rendering the retrovirus replication defective. The use of retroviruses for gene delivery to cells is described in more detail above.

It has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) Proc. Natl. Acad. Sci. USA 86:9079-9083; Julan et al. (1992) J. Gen. Virol. 73:3251-3255; and Goud et al. (1983) Virology 163:251-254); or coupling cell surface receptor ligands to the viral env proteins (Neda et al. (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). Thus, in a specific embodiment of the invention, viral particles containing a nucleic acid molecule containing a gene of interest operably linked to appropriate regulatory elements, are modified for example according to the methods described above, such that they can specifically target subsets of liver cells. For example, the viral particle can be coated with antibodies to surface molecule that are specific to certain types of liver cells. This method is particularly useful when only specific subsets of liver cells are desired to be ransfected.

Another viral gene delivery system useful in the present invention utilizes adenovirus-derived vectors. Recombinant adenoviruses can be advantageous in certain circumstances in that they are not capable of infecting nondividing cells. Furthermore, the virus particle is relatively stable and amenable to purification and concentration, and as above, can be modified so as to affect the spectrum of infectivity.

Yet another viral vector system useful for delivery of a nucleic acid molecule comprising a gene of interest for gene therapy is the adeno-associated virus (AAV), described in detail above. Other viral vector systems that may have application in gene therapy have been derived from herpes virus, vaccinia virus, and several RNA viruses.

Other methods relating to the use of viral vectors in gene therapy can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G.,(1994)Ann. N.Y Acad. Sci. 716:90-101; Strayer, D. S. (1999) Expert Opin. Invetig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

The pharmaceutical compositions can be included in a container, pack, or dispenser together with instructions for administration.

IX. Administration of CD28 Motif Modulating Agents

CD28 motif modulating agents of the invention are administered to subjects in a biologically compatible form suitable for pharmaceutical administration in vivo to either enhance or suppress T cell mediated immune response. By “biologically compatible form suitable for administration in vivo” is meant a form of the protein to be administered in which any toxic effects are outweighed by the therapeutic effects of the protein. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary to achieve the desired result. For example, a therapeutically active amount of a CD28 motif modulating agent may vary according to factors such as the disease state, age, sex, and weight of the individual, and the ability of peptide to elicit a desired response in the individual. Dosage regimen may be adjusted to provide the optimum therapeutic response. For example, several divided doses may be administered daily or the dose may be proportionally reduced as indicated by the exigencies of the therapeutic situation.

The therapeutic or pharmaceutical compositions of the present invention can be administered by any suitable route known in the art including for example intravenous, subcutaneous, intramuscular, transdermal, intrathecal or intracerebral or administration to cells in ex vivo treatment protocols. Administration can be either rapid as by injection or over a period of time as by slow infusion or administration of slow release formulation. For treating tissues in the central nervous system, administration can be by injection or infusion into the cerebrospinal fluid (CSF). When it is intended that an agent such as a polypeptide be administered to cells in the central nervous system, administration can be with one or more agents capable of promoting penetration of the polypeptide across the blood-brain barrier.

Active compounds of the present invention can also be linked or conjugated with agents that provide desirable pharmaceutical or pharmacodynamic properties. For example, the active compound can be coupled to any substance known in the art to promote penetration or transport across the blood-brain barrier such as an antibody to the transferrin receptor, and administered by intravenous injection. (See for example, Friden et al., 1993, Science 259: 373-377 which is incorporated by reference). Furthermore, the active compound can be stably linked to a polymer such as polyethylene glycol to obtain desirable properties of solubility, stability, half-life and other pharmaceutically advantageous properties. (See for example Davis et al., 1978, Enzyme Eng 4: 169-73; Burnham, 1994, Am J Hosp Pharm 51: 210-218, which are incorporated by reference).

Furthermore, the active compound can be in a composition which aids in delivery into the cytosol of a cell. For example, the peptide may be conjugated with a carrier moiety such as a liposome that is capable of delivering the peptide into the cytosol of a cell. Such methods are well known in the art (for example see Amselem et al., 1993, Chem Phys Lipids 64: 219-237, which is incorporated by reference). Alternatively, the CD28 polypeptide can be modified to include specific transit peptides or fused to such transit peptides which are capable of delivering the active compound into a cell. In addition, the polypeptide can be delivered directly into a cell by microinjection.

The compositions are usually employed in the form of pharmaceutical preparations. Such preparations are made in a manner well known in the pharmaceutical art, examples of which are described in further detail above. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The active compound can also be incorporated into a solid or semi-solid biologically compatible matrix which can be implanted into tissues requiring treatment.

Dose administration can be repeated depending upon the pharmacokinetic parameters of the dosage formulation and the route of administration used. It is also provided that certain formulations containing the CD28 polypeptide or fragment thereof are to be administered orally. Such formulations are preferably encapsulated and formulated with suitable carriers in solid dosage forms, as described above.

The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method for the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

An active compound which is polypeptide may be therapeutically administered by implanting into patients vectors or cells capable-of producing a biologically-active form of the polypeptide or a precursor of the polypeptide , i.e. a molecule that can be readily converted to a biological-active form of the polypeptide by the body. In one approach cells that secrete the polypeptide may be encapsulated into semipermeable membranes for implantation into a patient. The cells can be cells that naturally express the polypeptide or a precursor thereof, or the cells can be transformed to express the polypeptide or a precursor thereof. It is preferred that the cell and the polypeptide be of the same origin as the patient (e.g., human cells and human polypeptide for a human patient). The formulations and methods herein can be used for veterinary as well as human applications and the term “patient” or “subject” as used herein is intended to include human and veterinary patients.

Monitoring the influence of agents (e.g., drugs or compounds) on the activity of a CD28 motif can be applied not only in basic drug screening, but also in clinical trials. For example, the effectiveness of an agent determined by a screening assay as described herein to modulate CD28 motif activity, can be monitored in clinical trials of subjects. In such clinical trials, the activity of a CD28 motif can be used as a “read out”.

In a preferred embodiment, the ability of a CD28 motif modulating agent to modulate the activity of a CD28 motif can be measured by detecting an improvement in the condition of the patient after the administration of the agent. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

X. Kits of the Invention

Another aspect of the invention pertains to kits for carrying out the screening assays, modulatory methods or diagnostic assays of the invention. For example, a kit for carrying out a screening assay of the invention can include a cell comprising a polypeptide comprising a CD28 motif, means for determining CD28 motif activity and instructions for using the kit to identify-modulators of CD28 motif activity. In another embodiment, a kit for carrying out a screening assay of the invention can include a composition comprising a polypeptide comprising a CD28 motif, means for determining CD28 motif activity and instructions for using the kit to identify modulators of CD28 motif activity.

In another embodiment, the invention provides a kit for carrying out a modulatory method for the invention. The kit can include, for example, a modulatory agent of the invention (e.g., a CD28 survival or proliferation motif modulatory agent) in a suitable carrier and packaged in a suitable container with instructions for use of the modulator to modulate CD28 motif activity.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

This invention is further illustrated by the following examples which should not be construed as limiting. The contents of all references, patents, and published patent applications cited throughout this application, as well as the Figures and the sequence listing, are hereby incorporated by reference.

### EXAMPLES

The following materials and methods were used in the examples:

Mice: CD28-deficient mice on the DO11.10 TCR transgenic background were obtained from C. Thompson and S. Reiner (University of Pennsylvania, Philadelphia, Pa.). D011.10 mice were obtained from K. Murphy (Washington University, St. Louis, Mo. (3)). Balb/c mice were purchased from Jackson Laboratories (Bar Harbor, Me.).

Antibodies: Anti-CD3 (145-2C11, Hamster IgG) was provided by J. A. Bluestone (University of California, San Francisco, Calif.). Anti-CD28 (PV-1, Hamster IgG) was provided by C. June (University of Pennsylvania, Philadelphia, Pa.). Anti-Bcl-X_{L} antibodies (13.6, rabbit polyclonal IgG and clone 7B2, mouse IgG3) were provided by L. Boise (University of Miami, Miami, Fla.). All other mAb were purchased from PharMingen (San Diego, Calif.).

Rotroviral Infections. Full-length and mutant murine CD28 cDNA was cloned into the retroviral vectors GFPRV and CD4RV (provided by W. Sha, University of California, San Francisco and K. Murphy, Washington University, St. Louis, Mo.), to produce expression vectors encoding CD28 wild type, CD28dcyto, CD28d16, CD28Y170F, CD28P187,190A, and CD28Y188F. GFPRV encodes a bicistronc message for the cloned insert and GFP. The retroviral vectors were transiently transfected into the Phoenix Eco packaging cell line (provided by G. Nolan, Stanford University, Palo Alto, Calif.) as previously described (4, 5). Retroviral supernatants were incubated with activated splenocytes and expression of GFP and CD28 determined by flow cytometry. Infection efficiencies ranged from 10-20% between experiments, and were similar for all constructs within a given experiment. For Western blotting, the cells were infected with the CD4RV retrovirus (encoding for tailless human CD4 instead of GFP) and sorted by immunomagnetic beading using anti-human CD4 microbeads and an AutoMACS cell sorter (Miltenyi Biotec, Auburn, Calif.). Expression of CD28 was confirmed in all experiments by flow cytometry.

Proliferation assays: Wild type or CD28-deficient DO11.10 splenocytes were isolated and infected with retrovirus encoding for either vector alone, full-length CD28, or mutant CD28 proteins. 5×10⁴ infected cells were cocultured with 1.5×10⁵ T-depleted, irradiated splenocytes from Balb/c mice. Graded doses of OVA₍₃₂₃₋₃₃₉₎ peptide were added alone or in combination with murine CTLA4Ig (10 μg/ml, provided by Genetics Institute, Cambridge Mass.), anti-CD28 mAb (1.0 μg/ml) or control Ig. Proliferation was determined by tritiated thymidine incorporation for the final 8 hours of a 72 hour culture. All conditions were plated in quadruplicate and the mean +/− standard deviation of the quadruplicate wells was determined. Culture supernatants were harvested from replicate plates at 48 hours and were assayed for IL-2 content by CTLL-2 bioassay. All experiments were repeated a minimum of three times. Representative data from one experiment is presented.

Bcl-X_{L} expression: Retrovirally infected cells were enriched by immunomagnetic cell sorting and stimulated with immobilized anti-CD3 (10 μg/ml) alone or in combination with soluble anti-CD28 (1.0 μg/ml) for 48 hours. The cells were lysed in 0.2% NP40 lysis buffer, fractionated on a 12.5 % SDS-PAGE gel, and then transferred to a PVDF membrane, The membrane was then probed with anti-Bcl-X_{L} anti-sera and then developed by enhanced chemiluminescense (ECL). Membranes were reprobed with anti-actin antibody (Clone C4, Boehringer Mannheim, Indianapolis, Ind.) to verify equal protein loading. For intracellular flow cytometric analysis of Bcl-X_{L}, cells were surface stained with FITC conjugated anti-CD4 followed by intracellular staining with anti-Bcl-X_{L} mAb 7B2 or an isotype matched control antibody and analyzed on a FacsCalibur flow cytometer.

### Example 1

### CD28-mediated T cell activation and proliferation depends upon specific C-terminal proline residues

The most profound effect of CD28 mediated costimulation is the activation of naïve T cells (Lenschow, D. J.,et al., 1996. Annu Rev Immunol 14:233). The mechanism by which CD28 regulates proliferative and anti-apoptotic signaling pathways was investigated by reconstituting primary T cells which were deficient in CD28 (obtained from OVA specific TCR transgenic, CD28-deficient mice) with various mutants of CD28, by retroviral gene transfer. The OVA specific cells can be conveniently activated using the OVA₍₃₂₃₋₃₃₉₎ peptide antigen. This approach allowed the examination of the function of the CD28 mutants in a physiologic context, wherein primary cells were activated with peptide antigen presented by normal APC.

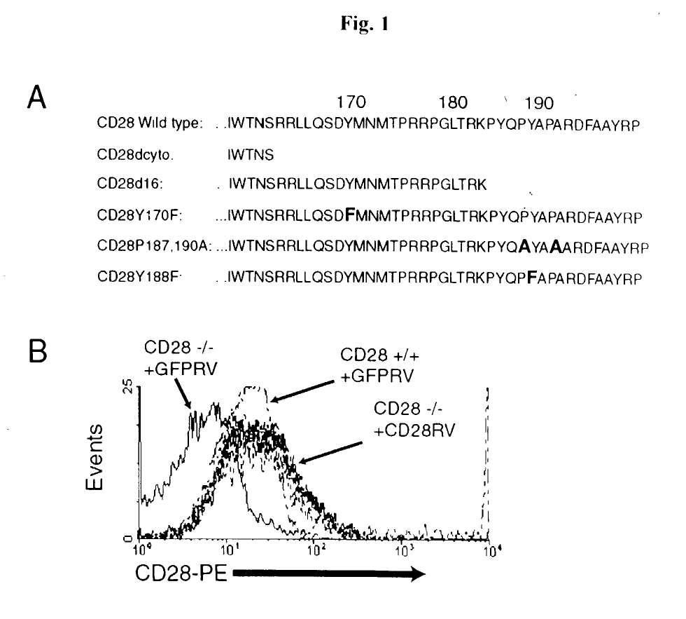

Expression of the CD28 proteins was confirmed by staining the T cells with PE-conjugated anti-CD28 mAb and two color flow cytometric analysis. As shown in FIG. 1, retroviral infection of the CD28-deficient T cells with the various CD28 mutants resulted in expression of the CD28 proteins on the cell surface at levels comparable to that of endogenous CD28 expressed on control infected splenocytes. The expression level of the retrovirally expressed mutant or wild type CD28 proteins were all similar, (shown as superimposed histograms labeled CD28 −/− + CD28RV).

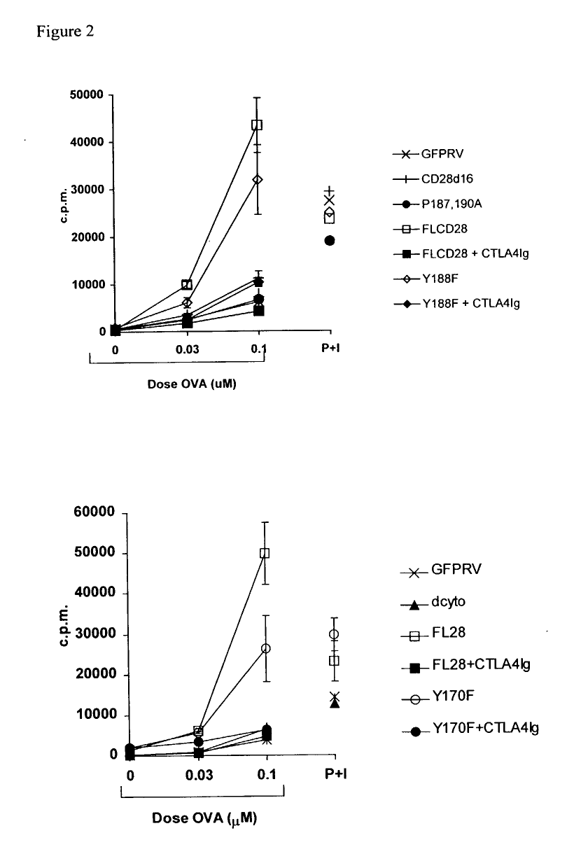

The T cells expressing the CD28 mutants were then analyzed for their ability to mount a proliferative response to antigen presented by normal APC. Wild type or CD28-deficient T cells infected with control retrovirus, or CD28-deficient T cells infected with full length or mutant CD28, were cocultured with T-depleted, irradiated splenocytes isolated from Balb/c mice. Peptide antigen was added either alone, with CTLA4Ig or a control Ig and proliferation determined by tritiated thymidine incorporation. The results presented in FIG. 2 indicate that reconstitution with wild type CD28 (FLCD28) resulted in a strong proliferative response. The proliferation was B7-dependent, as inclusion of CTLA4Ig in the cultures inhibited the proliferative response. Reconstitution with a mutant which lacked the cytoplasmic tail (dcyto) or the C-terminal 16 amino acids (CD28d16) did not produce any significant CD28-dependent proliferation.

Within the C-terminal region of CD28 is the sequence, PYAP, which may function to recruit and activate proteins which contain either SH2 or SH3 domains. Reconstitution with a CD28 mutant which contained alanine instead of proline residues at position 187 and 190 (CD28P 187,190A) abrogated CD28-dependent proliferation seen with CD28 wild type. Expression of a CD28 mutant which lacked the tyrosine (Y188F) within this motif had less effect, suggesting that proline mediated interactions with SH3 domains are critical for downstream signaling events.

CD28 also contains the motif YMNM in a position which is proximal to the membrane. Following phosphorylation of the tyrosine (Y170), CD28 can bind and activate PI 3-kinase (Pagès, F., et al., 1994. Nature 369.327). The role of PI 3-kinase activation in CD28 function remains controversial (Pages, F., et al., 1994. Nature 369:327; Hutchcroft, J. E., and B. E. Bierer. 1996. J Immunol 156.4071, Crooks, M. E., et al 1995. Mol Cell Biol 15:6820; Lu, Y., et al., 1995. Eur J Immunol 25.533; Truitt, K. E., et al., 1996. J Immunol 156.4539; Pages, F., et al., M. 1996. J Biol Chem 271.9403). Reconstitution with a mutant CD28 which contains a phenylalanine substituted for the tyrosine at position 170 (Y170F) in this motif resulted in a moderate defect in CD28-dependent proliferation with responses ranging from 60-80% proliferation of controls in multiple experiments (FIG. 2B).

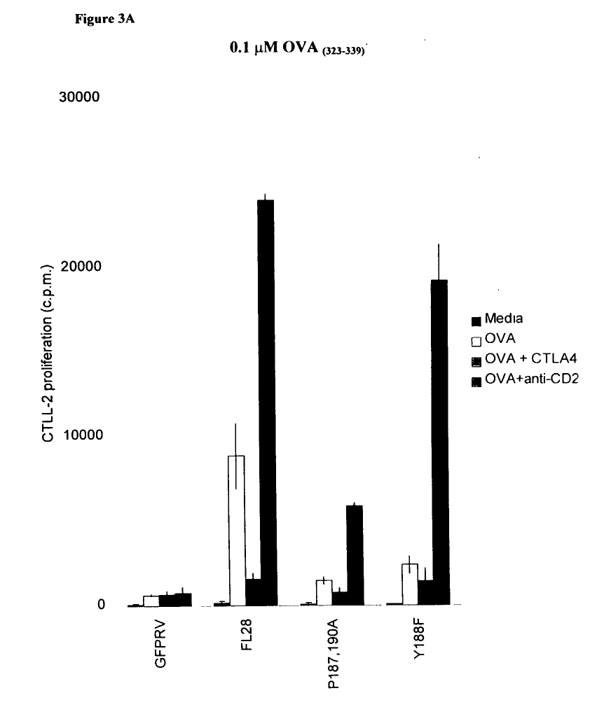

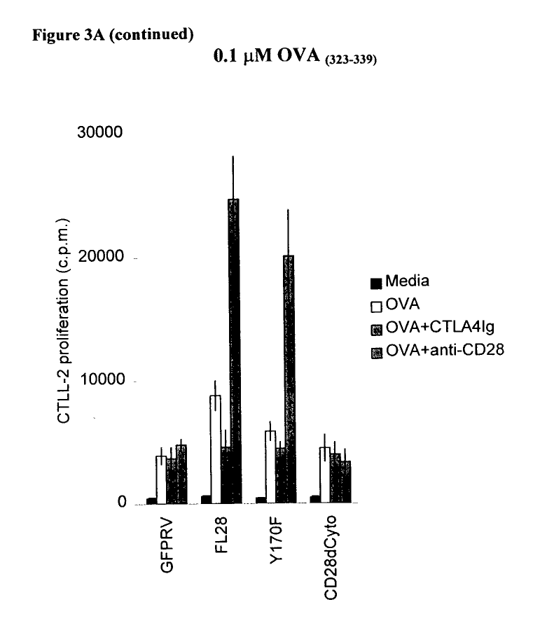

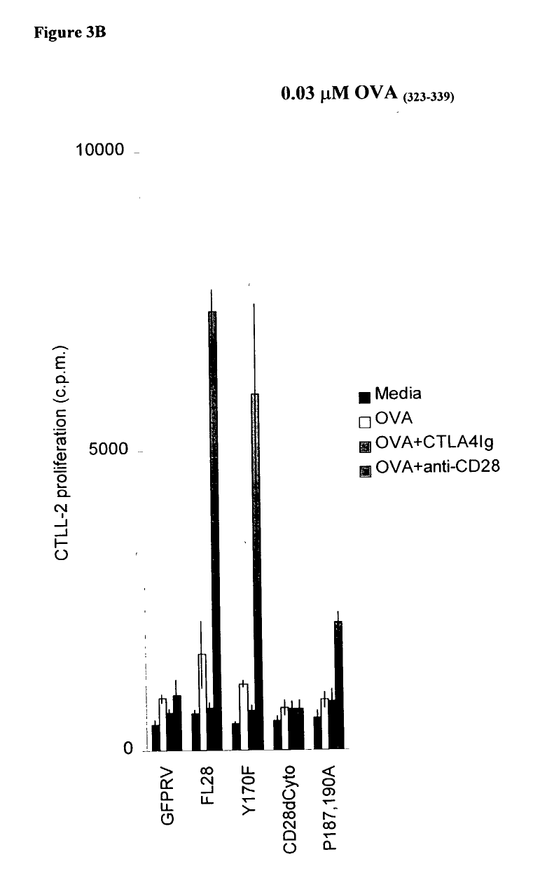

CD28 regulates IL-2 by both transcriptional and post-transcriptional processes (Fraser, J. D., etal., 1991. CD28. Science 251:313. Lindsten, T., et al 1989. Science 244:339). Following costimulation, IL-2 secretion is greatly enhanced. To assess the mechanism by which CD28 induced IL-2 secretion, CD28 deficient T cells expressing the exogenous CD28 wild type (FL28) and mutant proteins were stimulated with either OVA peptide antigen, or OVA peptide antigen in the presence of CTLA41g (to inhibit CD28 activation), or OVA peptide antigen in the presence of anti-CD28 (to activate CD28). Stimulation of cells expressing the CD28P187,190A mutant with antigen or antigen plus anti-CD28 led to a marked decrease in IL-2 secretion. In contrast, stimulation of the CD28Y188F and CD28Y170F mutants produced IL-2 levels comparable to wild type in response to anti-CD28 costimulation, suggesting these residues are not essential for CD28 regulation of IL-2 secretion. The IL-2 production in response to antigen alone was variable between experiments, with some experiments revealing little difference between CD28-deficient or CD28-sufficient T cells (compare FIGS. 3A and 3B). Inclusion of CTLA4Ig had little effect in these circumstances suggesting that endogenous B7-dependent costimulation was low. However, in all experiments, anti-CD28 mAb augmented IL-2 production from the CD28Y188F and CD28Y170F mutants to levels equivalent to FLCD28, whereas all other mutants had marked reduction in IL-2 secretion (FIG. 3). These results were observed using both a low and high dose of antigen (FIGS. 3A and 3B).

### Example 2

### Induction of Bcl-X_{L} requires residues important in PI 3-kinase activation

Costimulation through CD28 alters not only cell proliferation, but also cell survival (Noel, et al., 1996 #274). Activation of PI 3-kinase has been shown to regulate cellular processes important in cell survival and has been implicated in the regulation of Bcl-X_{L} (Collette, Y., D. et al., 1997. Eur J Immunol 27:3283. Jones, R. G., et al., 2000. J Exp Med 191:1 721. Marte, B. M., and J. Downward. 1997. Trends Biochem Sci 22:355). CD28 activated T cells have been shown to have a survival advantage by induction of the anti-apoptotic protein Bcl-X_{L} (Boise, L. H., et al., 1995. Immunity 3:87). To further investigate the role of CD28 activation in the regulation of Bcl-X_{L} the CD28 mutants were studied for their ability to induce BCl-X_{L} expression.

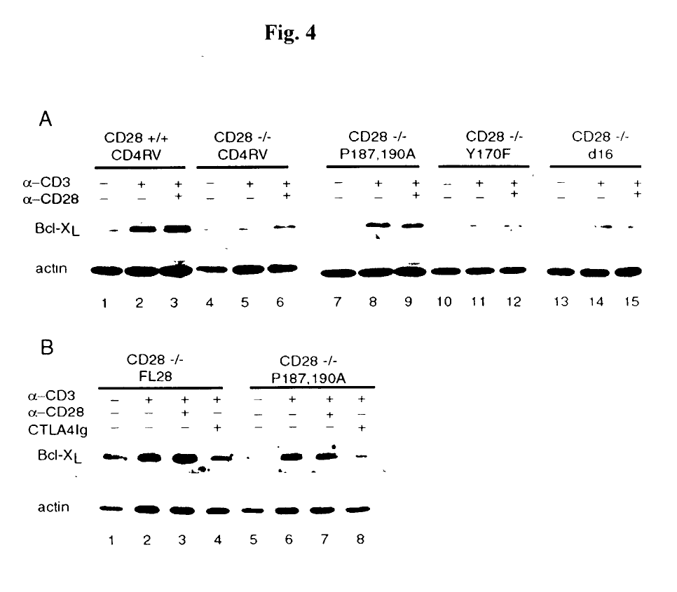

Retrovirally infected T cells were stimulated with anti-CD3 alone or in combination with anti-CD28, and 24 hours later lysed and examined for Bcl-X_{L} expression by western blot analysis (FIG. 4). Cells expressing wild type CD28 of either endogenous or retroviral origin demonstrated CD28-dependent induction of Bcl-X_{L} protein. The CD28Y 170F mutant was unable to induce Bcl-X_{L} induction after stimulation with anti-CD3 and anti-CD28 (FIG. 4). The tyrosine at position 170 of CD28 is a critical residue in PI 3-kinase activation by CD28 (Pagès, F., et al., 1994. Nature 369:327. Stein, P. H., et al., 1994. Mol Cell Biol 14.:3392). The CD28Y170F mutant was however, able to induce IL-2 secretion in response to CD28 crosslinking, at equivalent levels as cells reconstituted with wild type CD28. This suggests that induction of BCl-X_{L} by CD28 is not mediated exclusively by secondary effects of IL-2. Cell proliferation in response to stimulation appeared normal in the cells expressing the CD28Y170F mutant.

Anti-CD3 stimulation of cells reconstituted with the CD28PI 87,190A mutant led to increased Bcl-X_{L} expression. There was no apparent increase in expression when stimulated with anti-CD28, but this is likely due to the fact that B7-expressing cells present in the culture were providing endogenous costimulation. The endogenous costimulation was further indicated by the observation that CTLA4Ig effectively blocked the induction of Bcl-X_{L} by anti-CD3 in cells reconstituted with either FLCD28 or the CD28P187,190A mutant, thereby demonstrating intact B7-CD28 dependent expression of Bcl-X_{L} (FIG. 4B). The preservation of Bcl-X_{L} expression following stimulation of cells expressing the CD28P187,190A mutant was in contrast to the near complete abrogation of proliferation and IL-2 secretion observed in these same cells.

Deletion of the terminal 10 amino, acids of CD28 leads to a marked reduction in the binding of the p85 subunit of PI 3-kinase to CD28 (Pagès, F., et al., 1994. Nature 369:327. Pagès, F., et al., 1996. J Biol Chem 271:9403). Consistent with these data, the CD28 mutant that had a deletion of the C-terminal 16 amino acids (CD28d16) was also unable to induce Bcl-X_{L} induction. Bcl-X_{L} induction was induced in the CD28P187,190A mutant, suggesting other residues within this region are required. These data are consistent with a cooperative relationship between these two domains of CD28 in both the activation of PI 3-kinase and the induction of Bcl-X_{L}. Without being bound by theory, one model which accounts for these observations is the recruitment or activation of a kinase by a motif within the C-terminal 16 amino acids of CD28 which then phosphorylates the tyrosine at position 170, allowing for binding and activation of PI 3-kinase at this site.

### Example 3

### The Role of PI 3-kinase activation of PKB

To examine the role of PI 3-kinase in BCl-X_{L} expression using an approach complementary to the mutagenesis strategy, the effect of pharmacologic inhibition of PI 3-kinase activity was examined. Splenocyte cells from DO11.10 mice were stimulated with OVA₍₃₂₃₋₃₃₉₎ peptide either in media alone, or in the presence of the PI 3-kinase inhibitor LY294002 (25 μM) for 24 hours. CD69 and BCl-X_{L} expression was determined by staining with CD4-FITC followed by intracellular staining with anti-Bcl-X_{L} mAb, with subsequent analysis by flow cytometry.

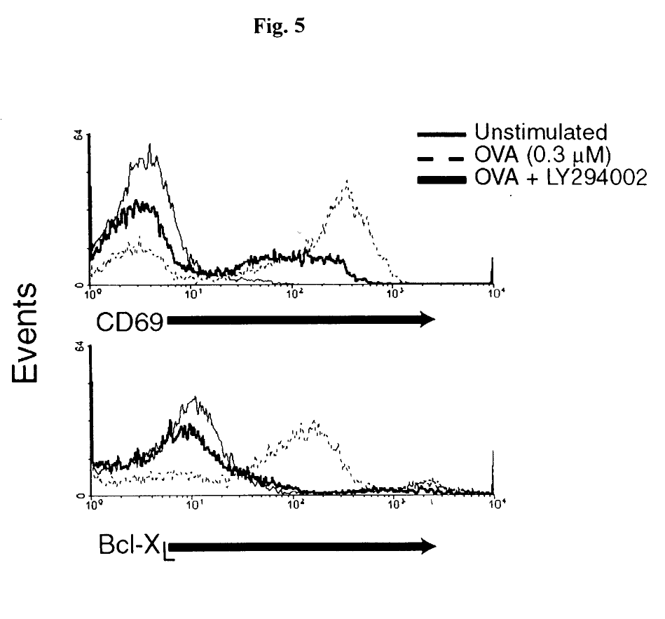

Treatment of wild type T cells with an inhibitor of PI 3-kinase (LY294002) resulted in a moderate decrease in expression of CD69, but complete blockade of Bcl-X_{L} induction (FIG. 5), in agreement with the results reported by Collette et al. using wortmannin (Collette, Y., et al., 1997. Eur J Immunol 27:3283). Together with the mutational data, these results indicate that the PI 3-kinase pathway is important for induction of Bcl-X_{L}.

Global inhibition of PI 3-kinase activity with pharmacologic inhibitors has been shown to inhibit T cell proliferation and IL-2 production (Shi, J., T. et al., 1997. J Immunol 158:4688. Eder, A. M., et al., 1998. J Biol Chem 273:28025). However, these reagents inhibit the activity of the enzyme and cannot selectively prevent activation by CD28 as opposed to other pathways. The decrease in CD69 expression observed following treatment with LY294002 is in fact consistent with an important role for PI 3-kinase in T cell activation. However, these mutational data imply that activation of PI 3-kinase by CD28 is not absolutely required for costimulation dependent proliferation and IL-2 production. Importantly, this does not suggest that PI 3-kinase is entirely dispensable for normal T cell activation and cytokine production.

Several groups have established that the tyrosine at position 170 is required for Pi 3-kinase binding and activation by CD28 (Pagès, F., et al., 1994. Nature 369:327. Stein, P. H., J. D. Fraser, and A. Weiss. 1994. Mol Cell Biol 14:3392). In addition, a second tyrosine based motif within the C-terminal 10 amino acids has also been shown to be involved in PI 3-kinase binding (Pages, F., et al. 1996. J Biol Chem 271. 9403). Thus, it is highly likely that these identical mutations in these retroviral constructs also fail to bind PI 3-kinase. However, it remains a formal possibility that these mutations interrupt other protein-protein interactions that are then responsible for the observed loss of Bcl-X_{L} induction.

The serine/threonine kinase protein kinase B (PKB, Akt) is a downstream effector of PI 3-kinase that is an important regulator of Bcl-X_{L} expression and is activated following CD28 ligation (Jones, R. G., et al., 2000. J Exp Med 191:1721, Parry, R. V., et al., 1997. Eur J Immunol 27:2495). These data are consistent with a mechanism in which CD28-dependent activation of PI 3-kinase leads to PKB activation and upregulation of Bcl-X_{L} expression.

Dahl et al. reported that overexpression of Bcl-X_{L} in CD28-deficient lymphocytes enhanced cell survival, but did not restore proliferation and cytokine secretion (Dahl, A. M., et al., 2000. J Exp. Med. 191:2031). Similarly, dissociation of CD28-dependent regulation of proliferation and expression of an important factor that effects cell survival was found. While this has not directly examined cell survival in this system, several groups have demonstrated that CD28 is a major regulator of Bcl-X_{L} expression in T cells, and that this is an important component in the control of cell survival (Sperling, A. I., et al., 1996. J Immunol 157:3909. Van Parijs, L., A. et al 1996. Immunity 4:321. Boise, L. H., et al., 1995. Immunity 387). Because retroviral infection requires activation of T cells for infection, the cells were not truly naive and therefore it is possible that these results may show a higher effect on Bcl-X_{L} expression than truly resting cells.

These examples show data from only a single time point for each parameter assayed. Peak proliferative responses and the greatest differences between constructs was observed at 72 hours. Similar trends were seen at earlier times for both proliferation and Bcl-X_{L} expression. At later time points, it is possible that the CD28Y170F mutation in particular might have had more of a proliferative defect, perhaps due to impaired survival given the failure to induce Bcl-X_{L}. Similarly, the CD28P187,190A mutation might have exhibited defective Bcl-X_{L} expression at later time points.

This analysis has enabled the examination of CD28 function on primary T cells following engagement of both CD28 and the TCR with native ligand expressed on normal APC. These data demonstrate that two of the consequences of CD28 ligation, proliferation and induction of Bcl-X_{L}, can be dissociated at the level of the amino acid sequence of CD28. The proline residues at position 187 and 190 of CD28 can recruit and activate Lck (Holdorf, A. D., et al., 1999. J Exp Med 190:375). Thus, proliferation induced by CD28 may be critically dependent upon this kinase. In contrast, mutation of a region of the cytoplasmic domain of CD28 known to be essential for PI 3-kinase activation had less effect on proliferation and IL-2 secretion, but prevented induction of Bcl-X_{L}. Pharmnacologic inhibition of PI 3-kinase suggests a key role for this enzyme in Bcl-X_{L} regulation, perhaps through activation of the PKB/Akt. Thus CD28 engages multiple signaling pathways that may independently regulate the biologic consequences of costimulation.

It is to be understood that the foregoing description and examples are intended to illustrate and not limit the scope of the invention. Other aspects, advantages and modifications within the scope of the invention will be apparent to those skilled in the art to which this invention pertains.

What is claimed is:

1. A method for selectively modulating T cell survival relative to T cell proliferation comprising contacting a T cell expressing a CD28 protein with an agent that selectively modulates the activity of a CD28 survival motif relative to the activity of a CD28 proliferation motif, to thereby selectively modulate survival of the T cell.

2. The method of claim 1, wherein said agent induces or enhances phosphorylation of the CD28 protein at a tyrosine residue which corresponds to the tyrosine at position 170 of the CD28 proteins set forth in SEQ ID NO:2.

3. The method of claim 1, wherein said contacting step results in a detectable increase in BCl-X_{L} expression in the T cell.

4. The method of claim 1, wherein said modulation of T cell survival is enhancement of T cell survival.

5. The method of claim 1, wherein said agent diminishes or interferes with phosphorylation of the CD28 protein at a tyrosine residue which corresponds to the tyrosine at position 170 of the CD28 protein set forth in SEQ ID NO:2.

6. The method of claim 1, wherein said contacting step results in a detectable decrease in Bcl-X_{L} expression in the T cell.

7. The method of claiml, wherein said modulation of T cell survival is reduction of T cell survival.

8. The method of claim 2, wherein said agent is a small molecule.

9. The method of claim 7 wherein the agent acts intracellularly to selectively inhibit or interfere with phosphorylation of the CD28 molecule at a tyrosine which corresponds to the tyrosine at position 170 of the CD28 protein set forth in SEQ ID NO:2.

10. The method of claim 9 wherein the agent is selected from the group consisting of: an intracellular antibody, a phosphatase, and a small molecule.

11. The method of one of claims 1-10, wherein said contacting step occurs in vivo.

12. The method of one of claims 1-10, v wherein said contacting step occurs in vitro.

13. A method for treating a subject having a condition that would benefit from selective enhancement of T cell survival relative to T cell proliferation comprising,
a) providing an agent that selectively increases the activity of a CD28 survival motif relative to the activity of a CD28 proliferation motif; and
b) administering the agent to the subject, to thereby selectively increase survival of the T cells of the subject relative to proliferation of the T cells of the subject.

14. The method of claim 13, wherein the condition is selected from the group consisting of: an immunosuppressive disorder, a condition that would benefit from survival of memory cells, and a neoplasia.

15. The method of claim 13, wherein said agent is selected from the group consisting of: an antibody and a small molecule that acts intracellularly to induce or enhance the phosphorylation of the CD28 protein at a tyrosine which corresponds to the tyrosine at position 170 of CD28 protein set forth in SEQ ID NO:2.

16. The method of claim 13, further comprising administering a second agent to the subject, wherein the second agent has immunostimulatory activity.

17. The method of claim 13 wherein the agent of step a) acts intracellularly on the T cells of the subject to induce or enhance the interaction of the CD28 survival motif with a CD28 survival motif binding partner.

18. The method of claim 17 wherein the CD28 survival motif binding partner is PI-3 kinase.

19. The method of claim 17 wherein the CD28 survival motif binding partner is selected from the group consisting of PI-3 kinase, Grb-2, and Gads.

20. The method of claim 17 wherein the agent of step a) acts intracellularly on the T cells of the subject to induce or enhance the phosphorylation of CD28 at a tyrosine which corresponds to the tyrosine residue at position 170 of the CD28 protein set forth in SEQ ID NO:2.

21. The method of claim 13, 17, or 20 wherein administering step b) results in a detectable increase in the expression of Bcl-X_{L} protein.

22. The method of claim 20 wherein the agent is selected from the group consisting of: an antibody and a small molecule.

23. A method for treating a subject having a condition that would benefit from selective reduction of T cell survival relative to T cell proliferation, comprising:
a) providing an agent that selectively decreases the activity of a CD28 survival motif relative to the activity of a CD28 proliferation motif; and
b) administering the agent to the subject to deliver the agent intracellularly to T cells of the subject, to thereby selectively decrease survival of the T cells of the subject relative to proliferation of the T cells of the subject.

24. The method of claim 23 wherein the agent of step a) acts intracellularly on the T cells of the subject to inhibit the interaction of the CD28 survival motif with a CD28 survival motif binding partner.

25. The method of claim 23 wherein the agent of step a) acts intracellularly on the T cells of the subject to diminish or interfere with the phosphorylation of a tyrosine residue at position 170 of the CD28 protein.

26. The method of claim 23, wherein administering step b) results in a detectable decrease in the expression of BCl-X_{L} protein in the T cells of the subject.

27. The method of claim 23, further comprising administering a second agent to the subject, wherein the second agent is an immunosuppressant.

28. The method of claim 25, wherein the agent of step a) is selected from the group consisting of: a phosphatase, an antibody, and a small molecule.

29. The method of claim 23, wherein the condition is selected from the group consisting of: a T cell neoplasia, a transplant-associated disorder, an allergic disease, graft-versus-host disease, and an autoimmune disease.

30. A cell-based method for identifying a compound which selectively modulates T cell survival relative to T cell proliferation, comprising:
a) providing a cell which expresses a CD28 protein that comprises a CD28 survival motif, in the context of a cell-based assay;
b) contacting the cell of step a) with a test compound; and
c) assaying for the ability of the test compound to modulate the phosphorylation of CD28 at a tyrosine residue which corresponds to the tyrosine at position 170 of the CD28 protein set forth in SEQ ID NO:2;
wherein a determination that the test compound modulates the phosphorylation of the CD28 protein, indicates that the test compound is a compound which selectively modulates T cell survival.

31. A cell-based method for identifying a compound which selectively modulates T cell survival relative to T cell proliferation comprising:
a) providing a cell which expresses a CD28 protein that comprises a CD28 survival motif, in the context of a cell based assay;
b) contacting the cell of step a) with a test compound; and
c) assaying for the ability of the test compound to modulate the activity or expression of Bcl-X_{L} in the cell;
wherein a determination that the test compound modulates the activity or expression of Bcl-X_{L} in the cell indicates that the test compound is a compound which selectively modulates T cell survival.

32. The cell-based method of claim 30 or 31 wherein the cell of step a) is a T cell.

33. A cell-free method for identifying a compound which selectively modulates T cell survival relative to T cell proliferation, comprising;
a) providing a polypeptide comprising a CD28 survival motif in the context of a cell-free assay system;
b) contacting the polypeptide with a test compound; and
c) assaying for the ability of the test compound to modulate phosphorylation of the CD28 survival motif at a tyrosine that corresponds to the tyrosine at position 170 of CD28 set forth in SEQ ID NO:2, by measuring a readout selected from the group consisting of:
(i) test compound binding to the region of the polypeptide comprising the tyrosine residue; and
(ii) test compound modulation of phosphorylation of CD28 survival motif at the tyrosine residue;
wherein detection of either (i) or (ii) indicates that the test compound is a compound that selectively modulates T cell survival.

34. A method for selectively modulating T cell proliferation relative to T cell survival comprising contacting a T cell expressing a CD28 protein with an agent that selectively modulates the activity of a CD28 proliferation motif relative to the activity of a CD28 survival motif, to thereby selectively modulate proliferation of the T cell relative to survival of the T cell.

35. The method of claim 34 wherein the agent selectively modulates the activity of a CD28 proliferation motif by modulating the interaction of prolines corresponding to the proline at position 187 and the proline at position 190 of the CD28 protein set forth in SEQ ID NO:2, with a molecule with which the CD28 proliferation motif interacts.

36. The method of claim 35 wherein said molecule is Lck protein.

37. The method of claim 34, wherein said contacting step results in an induction or enhancement of CD28 protein interaction with the SH3 domain of Lck protein.

38. The method of claim 34, wherein said contacting step results in a detectable increase in the expression of IL-2 protein.

39. The method of claim 34, wherein said modulation of T cell proliferation is enhancement of T cell proliferation.

40. The method of claim 34, wherein said contacting step results in a detectable diminishment or interference-with the interaction of the SH3 domain of Lck and the CD28 protein

41. The method of claim 34, wherein said contacting step results in a detectable decrease in the expression of IL-2 protein by the T cell.

42. The method of claim 34, wherein modulation of T cell proliferation is reduction in T cell proliferation.

43. The method of claim 35, wherein modulation of T cell proliferation is enhancement of T cell proliferation, and said agent is a small molecule which induces or increases said interaction.

44. The method of claim 36, wherein said modulation is inhibition of T cell proliferation, and said agent inhibits said interaction wherein said agent is selected from the group consisting of: an antibody and a small molecule.

45. The method of any one of claims 34-44, wherein the step of contacting occurs in vivo.

46. The method of any one of claims 34-44, wherein the step of contacting occurs in vitro.

47. A method for treating a subject having a condition that would benefit from selective increase of T cell proliferation relative to T cell survival comprising,
a) providing an agent that acts intracellularly to induce or enhance interaction of the prolines of CD28 protein which correspond to the proline at position 187 and the proline at position 190 of the CD28 protein set forth in SEQ ID NO:2 with a molecule with which the CD28 proliferation motif interacts; and
b) administering the agent to the subject to deliver the agent intracellularly to T cells of the subject, to thereby selectively increase proliferation of the T cells.

48. The method of claim 47, wherein said administering step results in a detectable increase in IL-2 protein production of the T cells of the subject.

49. The method of claim 47, wherein the agent of step a) is a small molecule, and the molecule of step a) is Lck.

50. The method of claim 47, further comprising administering a second agent to the subject wherein the second agent is an immunostimulant.

51. The method of claim 47, wherein the condition is selected from the group consisting of: a neoplasia, and an immunosuppressive disease.

52. A method for treating a subject having a condition that would benefit from selective reduction of T cell proliferation relative to T cell survival comprising,
a) providing an agent that acts intracellularly-to diminish or interfere with interaction of the prolines of the CD28 protein which correspond to the proline at position 187 and the proline at position 190 of the CD28 protein set forth in SEQ ID NO:2, with a molecule which interacts with a CD28 proliferation motif; and
b) administering the agent of step a) to the subject to deliver the agent intracellularly to T cells of the subject, to thereby selectively reduce proliferation of the T cells.

53. The method of claim 52, wherein administering step b) results in a detectable decrease in IL-2 production of the T cells of the subject.

54. The method of claim 52, wherein the molecule of step a) is Lck protein, and the agent of step a) is selected from the group consisting of: an intracellular antibody, a dominant negative mutant, an antisense oligonucleotide, and a small molecule.

55. The method of claim 52, further comprising administering a second agent to the subject, wherein the second agent is an immunosuppressant.

56. The method of claim 52, wherein the condition is selected from the group consisting of: a T cell neoplasia, an allergic disease, a transplant-associated disorder, graft-versus-host disease, and a lymphoproliferative disorder.

57. A cell-based method for identifying a compound which selectively modulates T cell proliferation relative to T cell survival, comprising:
a) providing a cell, which expresses a CD28 protein that comprises a CD28 proliferation motif, in the context of a cell-based assay;
b) contacting the cell of step a) with a test compound; and
c) assaying for the ability of the test compound to modulate the interaction of the prolines of the CD28 proliferation motif, which correspond to prolines 187 and 190 of the CD28 protein set forth in SEQ ID NO:2, with a molecule with which the CD28 proliferation motif interacts;
wherein a determination that the test compound modulates the interaction of the prolines of the CD28 proliferation motif with a molecule with which the CD28 proliferation motif interacts, indicates that the test compound is a compound which selectively modulates T cell proliferation relative to T cell survival.

58. A method of claim 57, wherein the assaying step c) comprises measuring the ability of the test compound to modulate the expression of IL-2 by the T cells.

59. A cell-free method for identifying a compound which selectively modulates T cell proliferation relative to T cell survival, comprising;
a) providing a polypeptide comprising a CD28 proliferation motif, in the context of a cell-free assay system;
b) contacting the polypeptide of a) with a test compound; and
c) assaying for the ability of the test compound to modulate the interaction of the prolines of the CD28 proliferation motif which correspond to prolines 187 and 190 of the CD28 protein set forth in SEQ ID NO:2, with a molecule with which the CD28 proliferation motif interacts;
wherein a determination that the test compound modulates the interaction of the prolines of the CD28 proliferation motif with a molecule with which the CD28 proliferation motif interacts, indicates that the test compound is a compound which selectively modulates T cell proliferation relative to T cell survival.
